(19) United States
(12) Patent Application Publication
(10) Pub. No.: US 2011/0270356 A1
(43) Pub. Date: Nov. 3, 2011

(54) POLYMERIC ENCAPSULATION OF MEDICAL DEVICE COMPONENTS

(76) Inventors: David Robert McKenzie, Artarmon (AU); Natalie Lisa James, Mosman (AU); John Raymond Grace, Newport Beach, (AU); Negin Amanat, Bronte (AU); Firas Awaja, Belfield (AU); Cedric Louis Edouard Chaminade, Newport Beach. (AU)

(21) Appl. No.: 13/036,843

(22) Filed: Feb. 28, 2011

Related U.S. Application Data
(60) Provisional application No. 61/308,442, filed on Feb. 26, 2010.

(51) Int. Cl. A61F 11/04; B32B 1/04; B32B 38/00; A61N 1/36

(52) U.S. Cl. 607/57; 428/76; 156/272.6

(57) ABSTRACT

A hermetic encapsulation and to a method of producing the same is provided. The hermetic encapsulation comprises a polymeric material disposed about a device component and has one or more plasma activated surfaces directly bonded to one another to hermetically seal the component within the material.

### CROSS-REFERENCE TO RELATED APPLICATIONS

This application claims priority from U.S. Provisional Patent Application No. 61/308,442 entitled “ENCAPSULATION,” filed on Feb. 26, 2010, which is hereby incorporated by reference herein in its entirety.

### BACKGROUND

1. Field of the Invention

The present invention generally relates to hermetic encapsulation, and more particularly, to polymeric encapsulation of medical device components.

2. Related Art

Implantable medical devices have provided benefits to recipients over recent decades. Implantable medical devices are devices having one or more components or elements that are at least partially implantable in a recipient. Implantable devices include active implantable medical devices that require power for operation, or passive medical devices that do not require power. Exemplary medical devices include, but are not limited, to hearing prostheses, such as hearing aids, cochlear implants, optically stimulating implants, middle ear stimulators, bone conduction devices, brain stem implants, direct acoustic cochlear stimulators, electro-acoustic devices and other devices providing acoustic, mechanical, optical, and/or electrical stimulation, cardiac pacemakers or monitor devices, neural stimulators or sensors, etc.

Implantable components of such medical devices generally require hermetic encapsulation for several reasons. First, the hermetic encapsulation isolates the implantable component(s) from the chemically aggressive in vivo environment. Second, the encapsulation protects surrounding tissues from exposure to any harmful materials leached from the implantable component(s) Hermetic encapsulation is particularly important for an active implantable device, where functionality may be compromised through the ingress of moisture or electrolytes.

Metals (e.g., titanium and its alloys) are conventionally used to encapsulate implantable medical device components. Metallic encapsulation can provide biocompatibility, low permeability to moisture and electrolytes, structural and dimensional stability and hermeticity, over the lifetime of an implantable medical device. However, metals tend to interfere with medical imaging technologies, such as magnetic resonance imaging (MRI) and computerized tomography (CT). Consequently, metallic encapsulation may produce artifacts that considerably degrade the quality of medical images acquired after an implantable medical device has been implanted.

Ceramics are also used to encapsulate components of implantable medical devices, particularly where a metallic encapsulation would interfere with the transmission of electrical signals to/from the device. The use of a ceramic encapsulation may significantly reduce the usable volume and the risk of foreign body reaction, as compared to metallic encapsulation. However, ceramic is inherently brittle, and thus vulnerable to impact-related failure. Moreover, high stress concentrations at the edges of the device can lead to cracks within the encapsulation, and hence ingress of fluids and egress of potentially harmful materials. Such occurrences may lead to catastrophic failure of the encapsulation.

### SUMMARY

According to one embodiment of the present invention an implantable medical device is provided. The device comprises: an implantable component, and a polymeric hermetic encapsulation disposed around the component having one or more plasma activated surfaces directly bonded to one another.

According to another embodiment of the present invention a method of hermetically encapsulating a component of an implantable medical device with a polymeric material is provided. The method comprises: exposing one or more surfaces of the polymeric material to plasma activation, positioning the polymeric material around the component such that portions of the one or more activated surfaces are abutting one another, and directly bonding the abutting portions of the one or more activated surfaces to one another to hermetically seal the component within the material.

### BRIEF DESCRIPTION OF THE DRAWINGS

Embodiments of the present invention will be described with reference to the following drawings, in which:

FIGS. 1a-L and 1a-R schematically illustrate the molecular mechanism of interdiffusion of polymer chains that may contribute to direct bonding between polymer surfaces in embodiments of the present invention;

FIGS. 1b-L and 1b-R schematically illustrate the molecular mechanism of entanglement of polymer chains that may contribute to direct bonding between polymer surfaces in embodiments of the present invention;

FIGS. 1c-L and 1c-R schematically illustrate the molecular mechanism of bridging of polymer chains that may contribute to direct bonding between polymer surfaces in embodiments of the present invention;

FIGS. 1d-L and 1d-R schematically illustrate the molecular mechanism of crystallisation of polymer chains that may contribute to direct bonding between polymer surfaces in embodiments of the present invention;

FIGS. 1e-1j schematically illustrate the diffusion of radical species, in accordance with embodiments of the present invention;

FIG. 2a is a schematic diagram of a cochlear implant in accordance with embodiments of the present invention, shown implanted in a recipient;

FIG. 2b is a perspective view of a hermetically encapsulated stimulator/receiver unit, in accordance with embodiments of the present invention;

FIG. 2c is a schematic view of plasma activation equipment that may be used in Example 1, in accordance with embodiments of the present invention;

FIG. 3a is a schematic side view illustrating the lap-shear joint geometry used in Example 1;

FIG. 3b is a schematic top view illustrating the lap-shear joint geometry used in Example 1;

FIG. 4 is a contour chart of stress failure (MPa) of plasma activated polyetheretherketone (PEEK) films as a function of plasma bias (kV) and plasma time (s):

FIG. 5a is a scanning electron microscopy (SEM) image of a PEEK film before plasma activation, taken at a first magnification;

FIG. 5b is a SEM image of the PEEK film of FIG. 5a, taken at a second magnification;

FIG. 6a is a SEM image, of a PEEK film after plasma activation using 5 kV and 300 s, taken at a first magnification;

FIG. 6b is a SEM image of the PEEK film of FIG. 6a, taken at a second magnification;

FIG. 6c is a SEM image of the PEEK film of FIG. 6a, taken at a third magnification;

FIG. 7a is a SEM image of a PEEK film after plasma activation using 10 kV and 150 s, taken at a first magnification;

FIG. 7b is a SEM image of the PEEK film of FIG. 7a, taken at a second magnification;

FIG. 7c is a SEM image of the PEEK film of FIG. 7a, taken at a third magnification;

FIG. 8a illustrates top and side views of a lap joint test sample used in Example 2;

FIG. 8b illustrates a laser welding set up used in Example 2;

FIG. 9a includes representative optical micrographs of semi-crystalline joints before mechanical testing;

FIG. 9b includes representative optical micrographs of amorphous joints before mechanical testing;

FIG. 10a illustrates a 2004a (amorphous; 20 W; 4 mm/s) sample having significant heat damage;

FIG. 10b illustrates a 2004a sample with damage at the joint edge indicated.

FIG. 11a is a graph illustrating mechanical testing results (mean lap-shear strength (LSS), MPa) for the amorphous morphology;

FIG. 11b is a graph illustrating mechanical testing results (mean lap-shear strength (LSS), MPa) for the semi-crystalline (right) morphology;

FIG. 12a illustrates post-failure SEM images of the weld interface in an amorphous weld;

FIG. 12b illustrates post-failure SEM images of the weld interface in a semi-crystalline weld;

FIG. 12c is an enlarged view of the inset shown in FIG. 12b;

FIG. 12d is an enlarged view of the inset shown in FIG. 12C;

FIGS. 13A-13H show optical micrographs and corresponding SEM images of two representative weld cross-sections: the amorphous weld (1004a) and the semi-crystalline weld (2008c);

FIG. 14 illustrates the plate configuration used in Example 3;

FIG. 15a is a side view of plates having an exemplary lap geometry used in Example 3 (the dimensions shown are in mm);

FIG. 15b is a front view of plates having an exemplary lap geometry used in. Example 3 (the dimensions shown are in mm);

FIG. 16 illustrates an exemplary laser weld location on the plates used in Example 3;

FIG. 17 includes various images of plates prior to mechanical testing.

FIG. 18 shows the underside (Lumogen containing plate) of a sample 7050_{—5 (}70 W and 50 s);.

FIG. 19a is a contour map of LSS (MPa) relative to power (W) and time (s);

FIG. 19b is a 3D surface plot of the contour map of FIG. 19a, viewed from a first angle;

FIG. 19c is a 3D surface plot of the contour map of FIG. 19a, viewed from a second angle;

FIG. 20a illustrates the dimensions of an exemplary individual film prior to bonding;

FIG. 20b illustrates sample dimensions (in mm) for direct and adhesive bonds;

FIG. 21a illustrates the dimensions of an exemplary individual film prior to bonding;

FIG. 21b illustrates the lap-shear configuration of two exemplary films;

FIG. 22a is a under view of a mounting technique for grip aids;

FIG. 22b is a top view of a mounting technique for grip aids;

FIG. 22c is a side view of a mounting technique for grip aids;

FIG. 22d shows a sample mounted with grip aids placed under tension;

FIG. 23 illustrates exemplary plate dimensions used in Example 5.

FIG. 24a illustrates first and second plates that may be utilized in Example 5;

FIG. 24b illustrates the first and second plates of FIG. 24a shown prior to bonding;

FIG. 24c illustrates the first and second plates of FIG. 24a after bonding;

FIG. 25a shows the placement of a sample on an O-ring, in accordance with Example 5;

FIG. 25b illustrates two nozzle positions used in accordance with Example 5;

FIG. 26a shows a sample holder that may be used in embodiments of the present invention;

FIG. 26b shows a measurement apparatus that may be used in embodiments of the present invention;

FIG. 27a is a schematic via of a capsule tested in accordance with Example 6;

FIG. 27b is a perspective view of the capsule tested in accordance with Example 6;

FIG. 28a is a cross-sectional view of a hermetic capsule, in accordance with embodiments of the present invention; and

FIG. 28b is a schematic top view of the capsule of FIG. 28a.

### DETAILED DESCRIPTION

Aspects of the present invention are generally directed to a polymeric encapsulation for components of an implantable medical device. In particular, a polymeric material having surface(s) treated via plasma activation is at least partially disposed around an implantable component. Portions of the plasma activated surface(s) are positioned abutting one another and are directly bonded through application of heat and/or pressure. In embodiments of the present invention, the polymeric encapsulation is a polyaryletherketone (PAEK) material.

In certain embodiments, the plasma activation may optionally be combined with plasma immersion ion implantation (PIII) and/or surface deposition to prepare the polymeric surfaces for direct bonding. For example, certain combinations of the above reduce the permeability of the resulting hermetic encapsulation.

Advantageously, embodiments of the present invention provide a reliable and permanent hermetic bond between the surfaces. Generally, embodiments of the present invention have advantages over metallic and ceramic encapsulations, including ease of fabrication, weight saving, flexibility, electrical and thermal insulation combined with electromagnetic transmission, and reduced manufacturing costs.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Documents referred to within this specification are included herein in their entirety by way of reference. Additionally, the reference to any prior art in this specification is not, and should not be taken as, an acknowledgment or any form of suggestion that that prior art forms part of the common general knowledge.

As mentioned above, aspects of the present invention relate to hermetic encapsulation and to methods of producing the same. As used herein, the phrase “hermetic encapsulation” refers to a polymer-based structure or layer that completely encloses an object, thereby inhibiting the ingress and egress of materials that may compromise the object and its surroundings, respectively. The object is typically, although not necessarily, a component of an active implantable medical device such as a hearing aid, cochlear implant, optically stimulating implant, middle ear stimulator, bone conduction device, brain stem implants, direct acoustic cochlear stimulator, electro-acoustic device, other device that provides acoustic, mechanical, optical and/or electrical stimulation, cardiac pacemaker or monitor device, neural stimulator or sensor, etc. Preferably, the hermetic encapsulation of the present invention exhibits a permeability to water vapor of less than 0.1 mg/m²/day, such as for example less than 0.01, 0.005, 0.001, 0.0005 or 0.0001 mg/m²/day.

As noted above, in certain embodiments, the hermetic encapsulation may be a PAEK material. The acronym “PAEK” is used herein to denote a family of semi-crystalline thermoplastics with excellent mechanical and chemical resistance properties that are generally retained to high temperatures. Polymers within the PAEK family include polyetherketone (PEK), polyetheretherketone (PEEK), polyetherketoneketone (PEKK), polyetheretherketoneketone (PEEKK) and polyetherketoneetherketoneketone (PEKEKK). In specific embodiments of the present invention, the hermetic encapsulation of the present invention comprises PEEK, the structure of which is shown below.

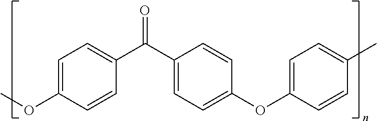

The Young's modulus and tensile strength of PFFK are approximately 3.7 GPa and 92 MPa, respectively. PEEK has a glass transition temperature around 143° C. and a melting point around 343° C. PEEK is highly resistant to thermal degradation as well as attack by both organic and aqueous environments.

The PAEK polymers are illustrative of the polymers that may be used for the encapsulation because they exhibit chemical resistance, mechanical robustness, and biocompatibility. Other polymers that may be used for the hermetic encapsulation include, but are not limited to polytetrafluoroethylene or other fluorinated polymers.

In embodiments of the present invention, the hermetic encapsulation may be a composite material that includes different polymeric materials. For example, a composite material may comprise a PAEK polymer in combination with one or more other polymers. Optional polymers include, but are not limited to, acrylonitrile butadiene styrene, celluloid, cellulose acetate, cycloolefin copolymer, ethylene chlorotrifluoroethlyene, ethylene tetrafluoroethylene, ethylene vinyl acetate, ethylene vinyl alcohol, fluorinated ethylene propylene, ionomer, Kydex, liquid crystal polymer, MP-1, perfluoroalkoxy, polyacetal, polyacrylonitrile, polyamide, polyamide-imide, polyamide-imide-phthalmide, polybutadiene, polybutylene, polybutylene terephthalate, polycaprolactone, polycarbonate, polychlorotrifluoroethylene, polycyclohexylene dim ethylene terephthalate, polyester, polyetherimide, polyethersulfone, polyethylene, polyethylenechlorinate, polyethylene terephthalate, polyhydroxyalkanoate, polyimide, polyketone, polylactic acid, polymethylmethacrylate, polymethylpentene, polyphenylene, polyphenylene oxide, polyphenylene sulfide, polyphenylene sulfone, polyphthalamide, polypropylene, polystyrene, polysulfone, polytetrafluoroethylene, polytrim ethylene terephthalate, polyurethane, polyvinyl acetate, polyvinyl chloride, polyvinylidene chloride, styrene-acrylonitrile and combinations thereof. Of these optional polymers, fluorinated ethylene propylene, MP-1, perfluoroalkoxy, polyamide-imide, polyamide-imide-phthalmide, polyetherimide and combinations thereof, are particularly preferred.

As noted above, during an encapsulation process in accordance with embodiments of the present invention, the surface of the polymer is exposed to plasma activation. The term “surface” is used herein to refer to the outer boundary of a polymer material, as well as the region 100 nm beneath the outer boundary.

The “direct bonding” of two polymer surfaces (that have been exposed to plasma activation) involves bringing the polymer surfaces together under pressure and/or heating (at a temperature that is below the polymer melting point, but often above the polymer glass transition temperature) to form a bond between the polymer surfaces. In certain embodiments, the bonding is referred to as autohesion. As used herein, autohesion is a formation of bond between similar polymer surfaces by applying pressure at a mildly elevated temperature.

It is believed that the direct bonding within the hermetic encapsulation of embodiments of the present invention arises by virtue of interdiffusion, entanglement and/or bridging of polymer chains between the polymer surfaces, or by the diffusion of radical species to the interface, leading to a cross linking bond across the interface.

Polymer chains are in a continual process of movement, even at ambient temperatures. This movement may include a diffusive motion (reptation), in which the polymer chains move significant distances relative to each other. Interdiffusion of polymer chains can therefore occur when two polymer surfaces are brought into intimate contact. Interdiffusion becomes more likely when the bonding temperature is higher than the polymer glass transition temperature.

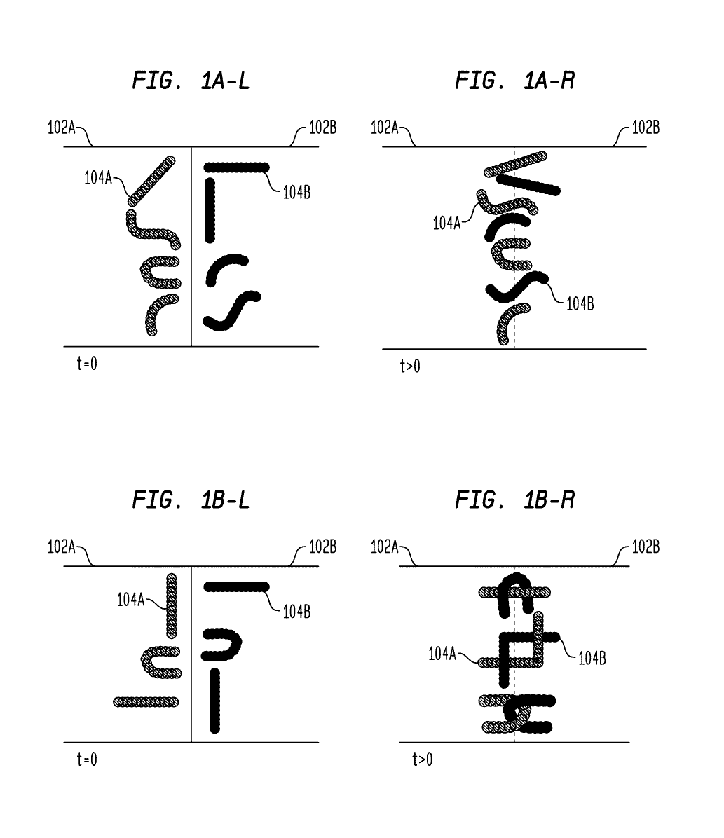

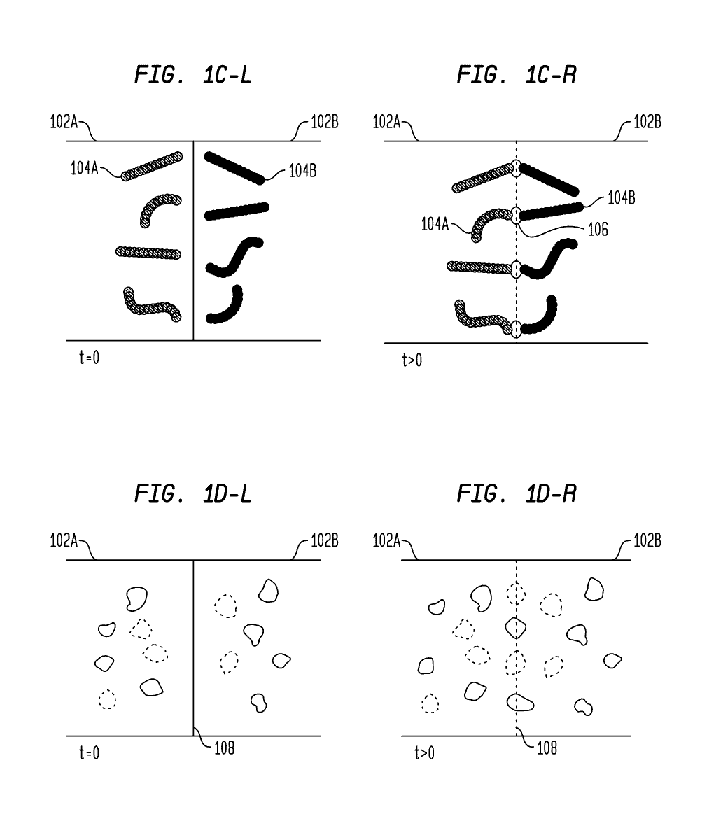

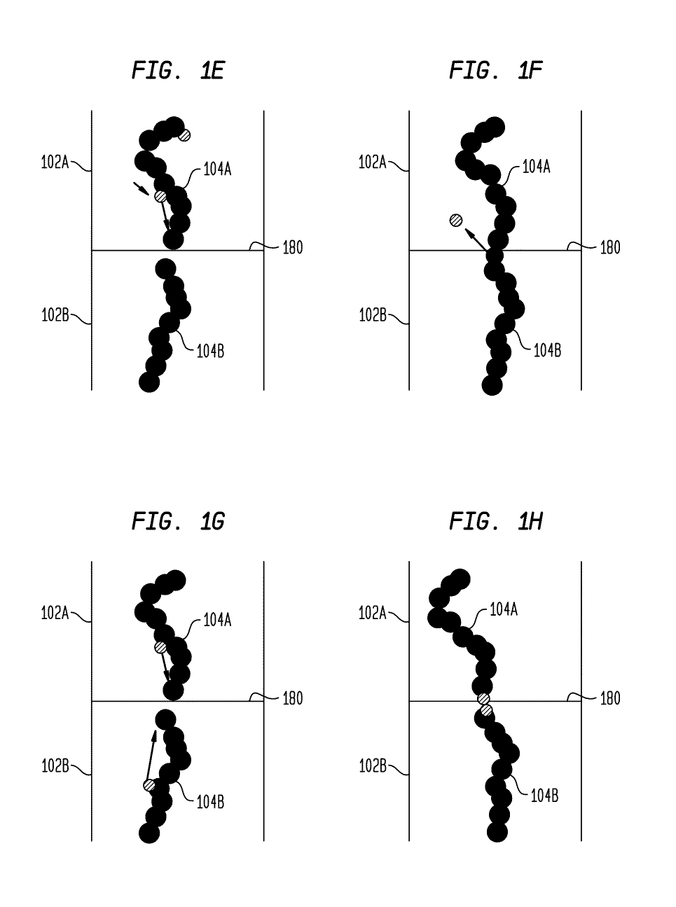

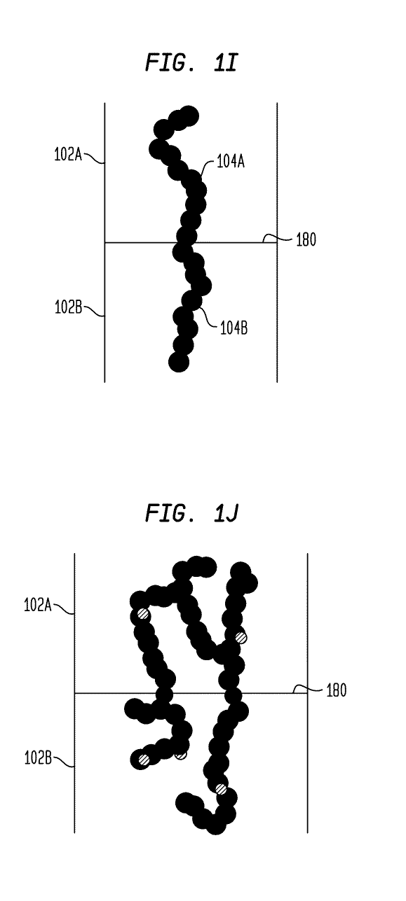

Interdiffusion is schematically illustrated in FIGS. 1a-L and 1a-R. Specifically, FIG. 1a-L illustrates two abutting polymer surfaces 102 that have just been brought into contact (time (t)=0). As shown, each surface has polymer chains 104. FIG. 1a-R illustrates surfaces 102 at a t>0. As shown, motion of polymer chains 104 have occurred such that some chains extend across to the opposing surface.

Entanglement of polymer chains between two polymer surfaces can occur after interdiffusion has occurred. Interdiffusion, and hence entanglement, is believed to increase with increasing polymer chain compactness. Polymer chain compactness may be controlled by the chemical conditions used to prepare the polymer surfaces (e.g., polymer molecular weight, pH, ionic strength) and/or by the degree of compacting force (if any) applied to the polymer surfaces.

Entanglement is schematically illustrated in FIGS. 1b-L and 1b-R. Specifically, FIG. 1b-L illustrates two abutting polymer surfaces 102 that have just been brought into contact (time (t)=0). As shown, each surface has polymer chains 104. FIG. 1b-R illustrates surfaces 102 at a t>0. As shown polymer chains 104 have become interdiffused and become entangled with chains from the opposing surface.

Bridging refers to the formation of covalent bonds between polymer chains when two polymer surfaces are brought into intimate contact (FIG. 1c). Such bonding occurs as a result of interactions between electrons and/or chemical groups present in the polymer surfaces.

Bridging is schematically illustrated in FIGS. 1c-L and 1c-R. Specifically, FIG. 1c-L illustrates two abutting polymer surfaces 102 that have just been brought into contact (time (t)=0). As shown, each surface has polymer chains 104. FIG. 1c-R illustrates surfaces 102 at a t>0. As shown, covalent bonds 106 have formed between polymer chains 104A and 104B.

The direct bonding within the hermetic encapsulation of embodiments of the present invention may be strengthened by crystallisation of the polymer chains following interdiffusion, entanglement and/or bridging. The rate, pattern and orientation of the crystal growth may be controlled to ensure cooling is sufficiently slow for crystallisation to occur and to enhance the direct bonding.

Crystallisation of the polymer chains is schematically illustrated in FIGS. 1d-L and 1d-R. Specifically, FIG. 1c-L illustrates two abutting polymer surfaces 102 that have just been brought into contact (time (t)=0). As shown, crystallisation of the polymer chains has occurred at the surface boundary after time t>0.

As noted above, another mechanism by which two polymer surfaces may be directly bonded is by the diffusion of unpaired electrons to the interface between the polymers. This diffusion of unpaired electrons, sometimes referred to as “radicals”, causes a cross linking reaction that forms a covalent bond across the interface. FIGS. 1e-1j schematically illustrate the diffusion of radical species, in accordance with embodiments of the present invention.

As shown in FIG. 1e, two polymer surfaces 102A, 102B are placed abutting, and polymer chains 104A, 104B are positioned on respective sides of the polymer interface 180. As shown in FIGS. 1f-1h, polymer chains 104 migrate to interface 180 and combine to generate a link that crosses the interface. In FIG. 1i, chains 104 form a covalent bond resulting in cross-linking of FIG. 1j.

As noted above, in embodiments of the present invention, plasma activation is used to treat polymer surfaces so as to facilitate direct bonding of treated polymer surfaces. The term “plasma” is used generally to describe the state of ionized gas. A plasma consists of charged ions (positive or negative), negatively charged electrons and neutral species. As known in the art, a plasma may be generated by combustion, flames, physical shock, or preferably, by electrical discharge, such as a corona or glow discharge. In radiofrequency (RF) discharge, a substrate to be treated is placed in a vacuum chamber and gas at low pressure is bled into the system. An electromagnetic field generated by a capacitive or inductive RF electrical discharge is used to ionize the gas. Free electrons in the gas absorb energy from the electromagnetic field and ionize gas molecules, in turn producing more electrons. A plasma “activates” a polymer surface by inducing molecular reactions in the polymer surface to render it more susceptible to bonding to another polymer surface. Such molecular reactions include, but are not limited to, scission, oxidation, nitration, crosslinking and/or condensation of polymer chains. These reactions can also include the formation of radical species that contain unpaired electrons.

It is believe that the effects of the plasma are caused by the bombardment by ions and, to a lesser extent the electrons and photons arising from the plasma,. It is believed that the primary effect of this bombardment is to cause the breakage of bonds in the polymer, leading to chain scission (shortening of the chains) and the formation of radicals (which are broken bonds containing unpaired electrons and are highly reactive). Some of the unpaired electrons are mobile and can diffuse, especially through the modified region. This allows radicals to diffuse to the surface for example, where they may react. It is believe that the secondary effects of the plasma activation are to create new bonds as crosslinks between chains (caused by for example the reaction of a radical with the neighbouring chains) and with environmental exposure to gases (including water vapour, oxygen and nitrogen) usually at the surface. The crosslinks can allow faster diffusion of unpaired electrons throughout the structure, for example, to the surface where they appear as radicals. The direct bonding may be assisted by the diffusion of unpaired electrons to the surface to form covalent bonds acting as cross links across the interface, or by the diffusion of broken polymer chains to the surface so that they move across the interface by diffusion. Chains that have been subject to scission may be more mobile (diffusive) than the original polymer chains.

Plasma activation in accordance with embodiments of the present invention may be conducted using a plasma activation apparatus, such as one incorporating a Helicon plasma source or other inductively or capacitively coupled plasma source. During activation, the apparatus is evacuated by attaching a vacuum nozzle to a vacuum pump. A suitable plasma forming gas from a gas source is bled into the evacuated apparatus through a gas inlet until the desired gas pressure in the apparatus and differential across the apparatus is obtained. An RF electromagnetic field is generated within the apparatus by applying current of the desired frequency to electrodes from an RF generator. Ionization of the gas in the apparatus is induced by the electromagnetic field, and the resulting plasma in the apparatus activates the polymer surfaces subjected to the plasma.

Suitable plasma forming gases used to activate the polymer surfaces include inorganic and/or organic gases. Inorganic gases are exemplified by helium, argon, nitrogen, neon, water vapor, nitrous oxide, nitrogen dioxide, oxygen, air, ammonia, carbon monoxide, carbon dioxide, hydrogen, chlorine, hydrogen chloride, bromine cyanide, sulfur dioxide, hydrogen sulfide, xenon, krypton, and the like. Organic gases are exemplified by methane, ethylene, benzene, formic acid, acetylene, pyridine, gases of organosilane, allylamine, organopolysiloxane, fluorocarbon and chlorofluorocarbon compounds, and the like. In addition, the gas may be a vaporized organic material, such as an ethylenic monomer to be plasma polymerized or deposited on the polymer surfaces. These gases may be used either singly or as a mixture of two or more, according to need. Preferred plasma forming gases according to the present invention are air, argon, hydrogen, methane, nitrogen and oxygen, more preferably, methane in combination with oxygen.

Typical plasma activation conditions include: a gas flow rate ranging from 1 sccm to 100 sccm, preferably from 35 sccm to 45 sccm; a pressure ranging from 0.1 Pa to 10 Pa, preferably from 1 Pa to 2 Pa; and a power ranging from 10 W to 1000 W; preferably from 100 W to 150 W.

In one embodiment of the present invention, the plasma activation involves plasma-immersion ion implantation (PIII), which serves to enhance the direct bonding between the polymer surfaces and/or further reduce the permeability of the hermetic encapsulation (see Example 1). Preferably, the PIII is conducted using ions derived from one or more of acetylene, argon, carbon, gold, hydrogen, iridium, methane, nitrogen, oxygen, parylene, platinum and titanium. More preferably, the PIII is conducted using ions derived from methane in combination with oxygen for optimum bonding. In certain embodiments, reduction of argon and/or hydrogen may facilitate permeability reduction. Typical PIII conditions include: a bias voltage ranging from 1 kV to 100 kV, preferably from 2 kV to 10 kV; and a time ranging from 10 s to 1000 s, preferably from 44 s to 300 s.

In another embodiment of the present invention, the polymer surfaces are exposed to surface deposition before, during and/or after the plasma activation to enhance the direct bonding between the polymer surfaces and/or further reduce the permeability of the hermetic encapsulation. That is, a metalized coating is provided to enhance the resistance of the enclosure to ingress of fluids and fluid vapor. Preferably, both the polymer surfaces that are to form the interior of the hermetic encapsulation and the polymer surfaces that are to form the exterior of the hermetic encapsulation are exposed to the surface deposition to achieve optimal reduction in permeability. The surface deposition is preferably conducted using materials derived from one or more of acetylene, argon, carbon, gold, hydrogen, iridium, methane, oxygen, parylene, platinum and titanium.

In a further embodiment of the present invention, the polymer surfaces are exposed to laser welding (e.g., transmission laser welding, laser butt welding) before, during and/or after the plasma activation. It is believed that the laser welding enhances the direct bonding by melting the polymer surfaces, which results in intermixing of polymer chains between the polymer surfaces.

During the laser welding, a laser beam is directed towards the overlapping polymer surfaces. The first polymer surface contacted by the laser beam allows the irradiation to pass through with a degree of transparency (the “transparent polymer surface”). Meanwhile, the second polymer surface contacted by the laser beam absorbs the irradiation due to the presence of one or more absorbing media therein or due to other modifications or characteristics thereof (the “absorbent polymer surface”). The irradiation absorbed by the absorbent polymer surface, combined with any irradiation absorbed by the transparent polymer surface, produces localized heating in the vicinity of the weld which leads to melting and bonding of the polymer surfaces at the interface.

An example of a suitable wavelength range in which the polymer surfaces may absorb laser light is from 800 nm to 2500 nm. The specific wavelength(s) of laser light absorbed by the absorbent polymer surface may be controlled through the type and concentration of absorbing media incorporated therein. Examples of absorbing media which may be used to modify the absorbent polymer surface include, but are not limited to, carbon black, Clearweld, Iriodin and Lumogen. Lumogen is the commercial name of a dye available from BASF AKTIENGESELLSCHAFT CORPORATION. A preferred absorbing medium is Lumogen, which is typically incorporated into the absorbent polymer surface at a concentration ranging from approximately 50 ppm to approximately 700 ppm, preferably from approximately 300 ppm to approximately 500 ppm. A PEEK surface doped with such levels of Lumogen, for example, will absorb laser light at a wavelength of approximately 1080 nm.

The laser welding may be conducted in a number of different modes, each of which involves a distinct set of irradiation conditions: single pass irradiation (see Example 2) typically involves a power ranging from approximately 1 W to approximately 100 W and a scan speed ranging from approximately 1 mm/s to approximately 100 mm/s; quasi-simultaneous irradiation (see Example 3) typically involves a power ranging from approximately 1 W to approximately 200 W, a scan speed ranging from approximately 1 mm/s to approximately 30,000 mm/s and an exposure time ranging from approximately 10 s to approximately 100 s; continuous irradiation typically involves a fluence rate ranging from approximately 1 W/mm² to approximately 20 W/mm2 and an exposure time ranging from approximately 0.1 s to approximately 100 s. It should be appreciated that these ranges are merely illustrative and do not limit embodiments of the present invention. For example, single pass irradiation may be conducted at different powers, such as approximately 9.2 W. Quasi simultaneous welding at 9.2 W may also be used. Additionally, the various ranges and examples are not limited to particular laser wavelengths, spot sizes, energy distributions. Polymer shapes, thicknesses, structures, surface conditions, etc.

Bond strength and hermeticity are key indicators of the quality of a bonded interface. Embodiments of the present invention also encompass a number of experimental protocols that may be used to measure, or predict, the performance of these properties over time (see Examples 4 to 6).

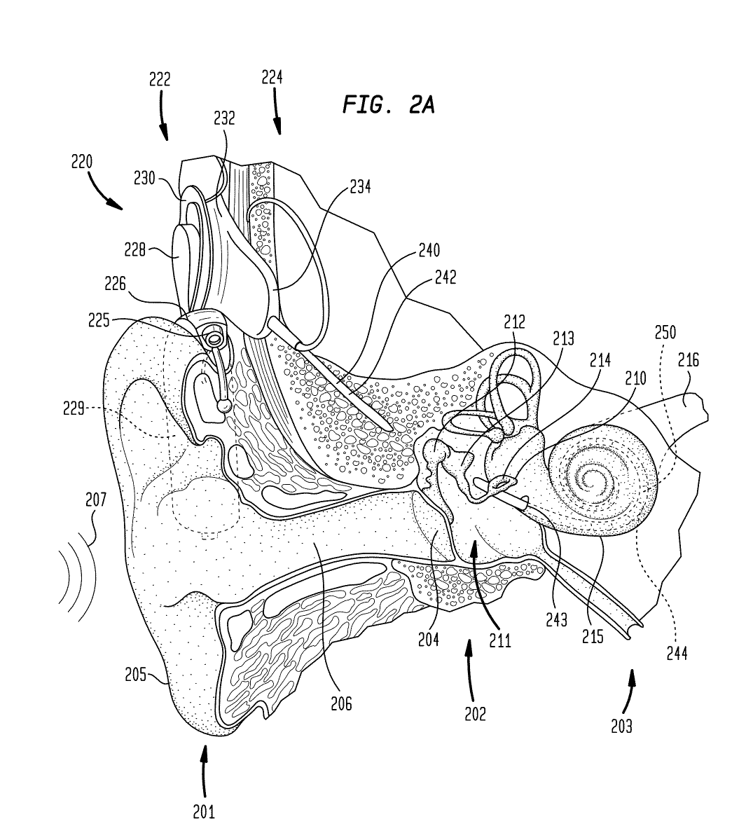

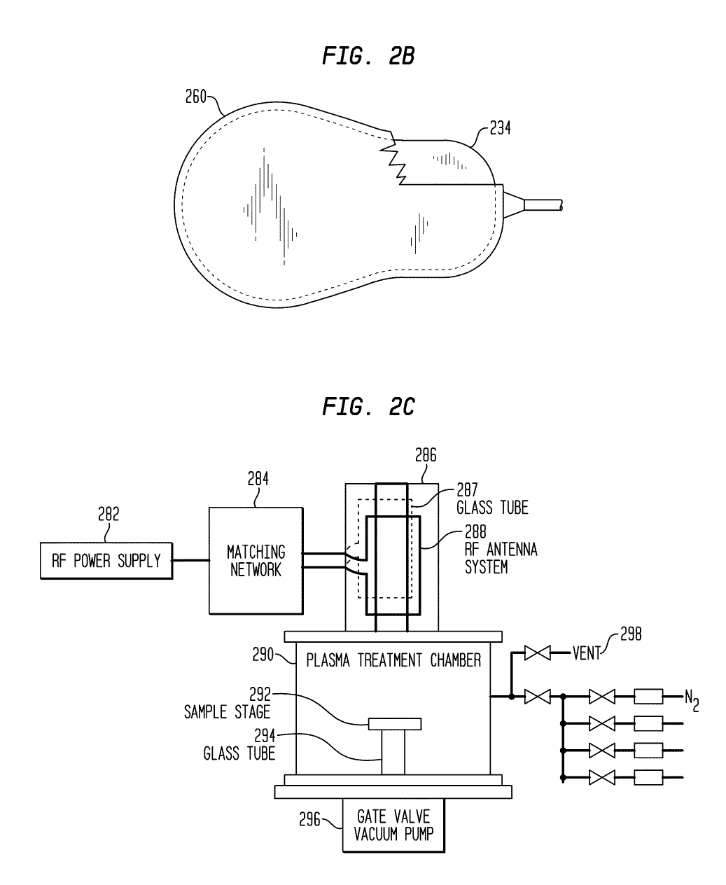

As noted above, implantable devices include, but are not limited to, hearing prostheses such as hearing aids, cochlear implants, optically stimulating implants, middle ear stimulators, bone conduction devices, brain stem implants, direct acoustic cochlear stimulators, electro-acoustic devices and other devices providing acoustic, mechanical and/or electrical stimulation, cardiac pacemakers or monitor devices, neural stimulators or sensors, etc. FIG. 2a is schematic diagram of an exemplary cochlear implant 220 in which embodiments of the present invention may be advantageously implemented.

In fully functional human hearing anatomy, outer ear 201 comprises an auricle 205 and an ear canal 206. A sound wave or acoustic pressure 207 is collected by auricle 205 and channeled into and through ear canal 206. Disposed across the distal end of ear canal 206 is a tympanic membrane 204 which vibrates in response to acoustic wave 207. This vibration is coupled to oval window or fenestra ovalis 210 through three bones of middle ear 202, collectively referred to as the ossicles 211 and comprising the malleus 212, the incus 213 and the stapes 214. Bones 212, 213 and 214 of middle ear 202 serve to filter and amplify acoustic wave 207, causing oval window 210 to articulate, or vibrate. Such vibration sets up waves of fluid motion within cochlea 215. Such fluid motion, in turn, activates tiny hair cells (not shown) that line the inside of cochlea 215. Activation of the hair cells causes appropriate nerve impulses to be transferred through the spiral ganglion cells and auditory nerve 216 to the brain (not shown), where they are perceived as sound. In certain profoundly deaf persons, there is an absence or destruction of the hair cells. Cochlear implants such a cochlear implant 220 is utilized to directly stimulate the ganglion cells to provide a hearing sensation to the recipient.

FIG. 2a also shows the positioning of cochlear implant 220 relative to outer ear 201, middle ear 202 and inner ear 203. Cochlear implant 220 comprises external component assembly 222 which is directly or indirectly attached to the body of the recipient, and an internal component assembly 224 which is temporarily or permanently implanted in the recipient. External assembly 222 comprises a sound input element, such as a microphone 225 for detecting sound which is output to a behind-the-ear (BTE) speech processing unit 226 that generates coded signals which are provided to an external transmitter unit 228, along with power from a power sburce 229 such as a battery. External transmitter unit 228 comprises an external coil 230 and, preferably, a magnet (not shown) secured directly or indirectly in external coil 230.

Internal component assembly 224 comprise an internal coil 232 of a stimulator unit 234 that receives and transmits power and coded signals received from external assembly 222 to other elements of stimulator unit 234 which apply the coded signal to cochlea 215 via an implanted electrode assembly 240. Electrode assembly 240 enters cochlea 215 at cochleostomy region 243 and has one or more electrodes 250 positioned on an electrode array 244 to be substantially aligned with portions of tonotopically-mapped cochlea 215. Signals generated by stimulator unit 234 are typically applied by an array 244 of electrodes 250 to cochlea 215, thereby stimulating auditory nerve 216.

FIG. 2b is a perspective view of stimulator unit 234 of having a hermetic encapsulation 260 in accordance with embodiments of the present invention positioned thereon. For illustrative purposes, a section of encapsulation 260 has been omitted from FIG. 2b.

Embodiments of the present invention will now be further described, by way of example only, with reference to the following non-limiting examples.

### Example 1

### Plasma Activation (Including PIII) of PEEK Films

### Materials

In this illustrative embodiment, semi-crystalline PEEK films having a thickness of 500 μm are used to create a polymeric encapsulation of a medical device component. The exemplary PEEK films that may be used have a 32% crystallinity, as measured by temperature modulated differential scanning calorimetry. The PEEK films have glossy and matt sides, but only the glossy sides were examined and used for bonding. The glossy sides showed no crystals on the surface, as measured by grazing incidence x-ray diffraction (GIXRD) and scanning electron microscopy (SEM), which indicated the presence of a thin amorphous layer over a crystalline sub layer.

### Plasma Activation

In this exemplary embodiment, plasma activation may be carried out in a plasma device, schematically illustrated in FIG. 2. The plasma device, shown as device 280, comprises a RF power (13.56 MHz) supply 282 capacitively coupled via an impedance matching network 284 to a plasma treatment chamber 286 using an externally mounted antenna system 288 and a glass tune 287. The diameter of the plasma chamber 286 is 100 mm and the plasma chamber is coupled to a stainless steel treatment chamber 290 of diameter 450 mm. The sample holder consists of a stainless steel plate 292 mounted on a glass tube 294, which is electrically isolated from the chamber and therefore can be biased or left at floating potential. The sample holder is mounted in the treatment chamber. The base pressure of the chamber was 7×10⁻³ Pa. The operating pressure range was 0.63 to 0.66 Pa. The flow rates of gases were 38.6 sccm for methane and 38.6 sccm for oxygen. The operating power of the RF power supply 282 was 100 to 150 W, with 25 to 50 W reflected power. In the case where the sample was biased, the holder was connected to a pulsed power supply delivering DC pulses of 2 to 10 kV at a frequency of 2800 Hz.

The exemplary PEEK films each have 100×100 mm sizes and the surfaces are initially cleaned using ethanol. In operation, the samples are placed on the sample holder in the plasma chamber and the system is evacuated using pump 296 to base pressure. After plasma activation, the chamber is vented via vent(s) 298 with air and samples were taken out for further experimental use.

### PIII

Two variables are selected based on preliminary observations and trials. The voltage (X1) measured in kV and treatment time (X2) measured in s. Hydrogen gas was used as the plasma medium. Table 1 shows the relationship between the experimental design code and the real values for each variable, and Table 2 shows exemplary experimental data.

TABLE 1
Relationship between experimental design code and real
values for voltage (X1) and treatment time (X2)

| | Coded | | | | |
|---|---|---|---|---|---|
| Real | −1.414 | −1 | 0 | 1 | 1.414 |
| X1 (kV) | 2 | 2.9 | 5 | 7.1 | 10 |
| X2 (s) | 0 | 44 | 150 | 256 | 300 |

TABLE 2
Experimental data

| Exp. | X1 | X2 | X1 real | X2 real |
|---|---|---|---|---|
| 1 | −1 | −1 | 2.9 | 44 |
| 2 | −1 | 1 | 2.9 | 256 |
| 3 | 1 | −1 | 7.1 | 44 |
| 4 | 1 | 1 | 7.1 | 256 |
| 5 | 0 | −1.414 | 5 | 0 |
| 6 | 0 | 1.414 | 5 | 300 |
| 7 | −1.414 | 0 | 2 | 150 |
| 8 | 1.414 | 0 | 10 | 150 |
| 9 | 0 | 0 | 5 | 150 |
| 10 | 0 | 0 | 5 | 150 |
| 11 | 0 | 0 | 5 | 150 |

### X-Ray Photoelectron Spectroscopy (XPS)

XPS measurements may be performed using a Specs XPS spectrometer (Specs GmbH, Germany), equipped with a monochromatised X-ray source (Al Ka, hu=1486.6 eV) operating at 200 W. The spectrometer energy scale is calibrated using the Au 4f_{7/2} photoelectron peak at binding energy E_{B}=83.98 eV. Survey spectra (average of 10 scans) are acquired for binding energies in the range 0 to 1400 eV, using a pass energy of 30 eV. C 1 s, 0 1 s and N 1 s region spectra were acquired at a pass energy of 23 eV to obtain higher spectral resolution. Peaks are fitted with synthetic Gaussian (70%)-Lorentzian (30%) components using the Marquardt-Levenberg fitting procedure in CasaXPS and are quantified using relative sensitivity factors supplied by the spectrometer manufacturer. Linear background subtraction was used and the spectra are charge corrected by setting the C 1 s CC/H component to 285.0 eV.

### Contact Angle Measurements

Contact angle measurements for three different solvents (deionized water, diiodomethane and formamide) may be performed following the sessile drop method with Kruss contact angle equipment DS 10. The contact angle values are calculated from the mean of the left and right hand side contact angles of the sessile drop. This was repeated five times across the surface of each sample; average contact angle values for each sample are then calculated. The surface free energies of the PEEK films are calculated using the Wu harmonic mean theory.

### SEM

Surface morphology characterization may be conducted by using a field emission SEM (FESEM, Zeiss ULTRA plus) with an operating voltage of 3 kV. The PEEK films are placed onto aluminum sample stubs by using conductive double sided carbon tape. In order to produce conductive surfaces, samples are coated with platinum in an Emitech K550X sputter coater at 25 mA for 2 minutes, which delivered an 8 nm thickness of platinum coating. Hot press

The PEEK films are cut into rectangular strips, 35×10 mm², using conventional scissors. Two rectangular strips are self-bonded together in the lap-shear joint geometry with a contact area of 10×10 mm². The bonding process was carried out at 200° C. and 3 MPa for a continuous 4 hr period with a Moore press (George E Moore & Sons, Birmingham Ltd., England).

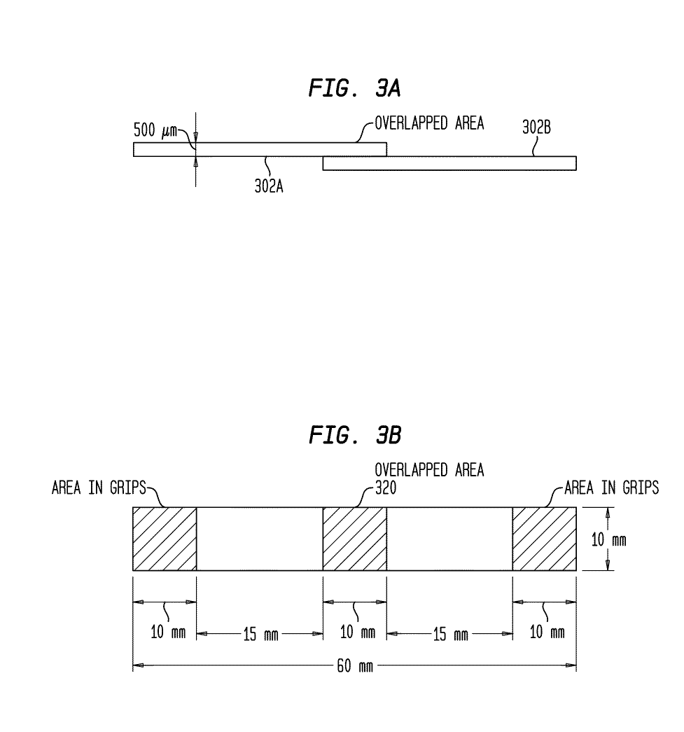

FIG. 3a is a schematic side view illustrating the lap-shear joint geometry used in Example 1, while FIG. 3b is a schematic top view of the geometry. As shown, the PEEK films 302 overlap one another by in region 320.

### Lap-Shear Test

Bonding strength values developed at the interface of PEEK lap-shear joints are measured with an Instron 5567 at a crosshead speed of 2 mm/min with a 1 kN load cell at room temperature. Force-displacement curves are recorded. Shear stress was calculated as the measured force at break, divided by the contact area and 5 replicates are tested to calculate an average value of the lap-shear strength a, neglecting the fact that the stress distribution over the overlapped area is non-uniform and higher at the ends. Results

Table 3 shows the stress failure results.

TABLE 3
Stress failure results

| | Bias | Plasma | Stress | | |
|---|---|---|---|---|---|
| | voltage | time | failure | Std. | |
| Exp. | (kV) | (s) | (MPa) | dev. | Failure |
| 1 | 2.9 | 44 | 3.28 | 0.31 | Cohesive at the bonding area |
| 2 | 2.9 | 256 | 4.45 | 0.23 | Cohesive at the bonding area |
| 3 | 7.1 | 44 | 2.41 | 0.24 | Cohesive at the bonding area |
| 4 | 7.1 | 256 | 3.95 | 0.24 | Cohesive at the bonding area |
| 5 | 5 | 0 | 0.50 | 0.02 | Adhesive |
| 6 | 5 | 300 | 2.50 | 0.7 | Cohesive at the bonding area |
| 7 | 2 | 150 | 3.78 | 0.44 | Cohesive at the bonding area |
| 8 | 10 | 150 | 5.00 | 0.07 | Failure at the substrate |
| 9 | 5 | 150 | 3.81 | 0.8 | Cohesive at the bonding area |
| 10 | 5 | 150 | 3.36 | 0.45 | Cohesive at the bonding area |
| 11 | 5 | 150 | 3.80 | 0.13 | Cohesive at the bonding area |

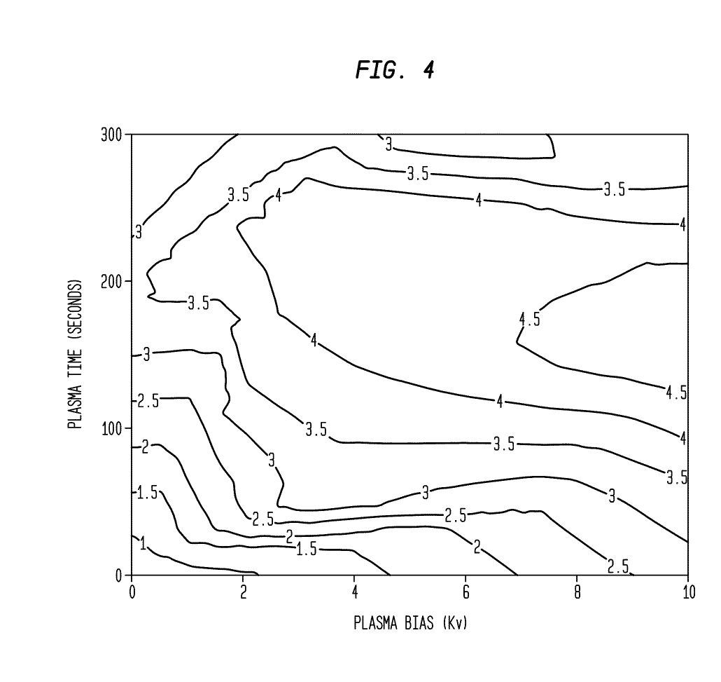

FIG. 4 shows a contour chart of stress failure (MPa) of plasma activated PEEK films as a function of plasma bias (kV) and plasma time (s).

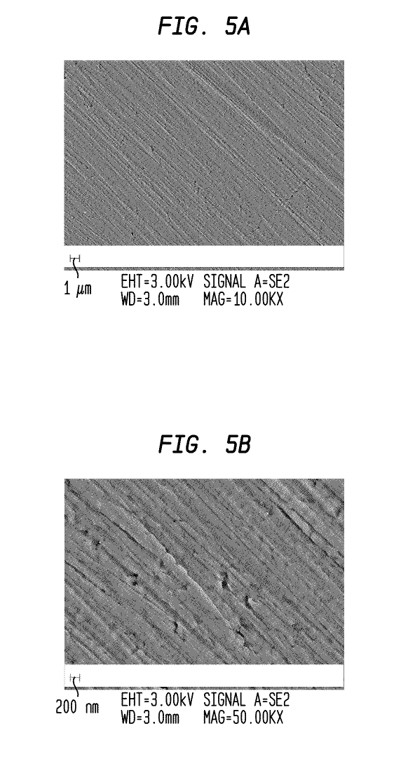

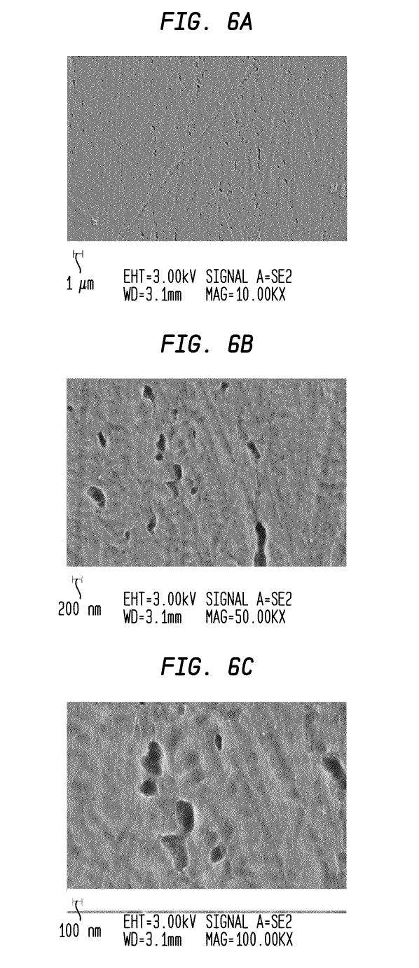

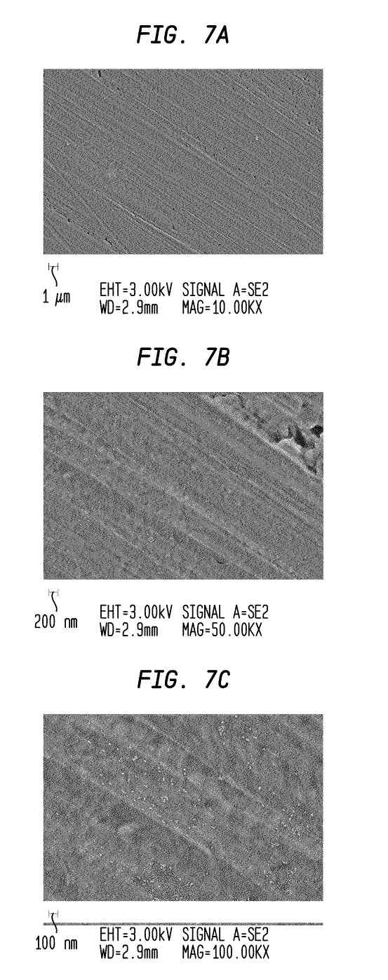

FIGS. 5a to 7c show SEM images (at different magnifications) of PEEK films: before plasma activation (FIG. 5); after plasma activation using 5 kV and 300 s (FIG. 6); and after plasma activation using 10 kV and 150 s (FIG. 7).

### Example 2

### Transmission Laser Welding (Single Pass Irradiation) of PEEK Films

### Materials

In this illustrative example, APTIV PEEK films of thickness 250 μm are utilized. Two morphologies of unfilled PEEK are obtained: amorphous (250a) and semi-crystalline (250c). A characteristic of amorphous PEEK is that it is relatively translucent to visible light, while semi-crystalline PEEK is not.

The infrared absorbing medium, Clearweld (Gentex Corporation, Supplier: Plastral, Australia), was obtained in liquid form, and applied to the surface of the interface to be lasered. To aid in sample-to-sample uniformity of the Clearweld application, the same operator conducted this process for all samples. Clearweld coatings are formulated for 940 nm to 1064 nm wavelength lasers and are used in applications where both the top and bottom polymers are laser transmissive.

Certain embodiments of the present invention use Lumogen (dispersed within the PEEK during molding), rather than Clearweld applied to the surfaces. PEEK having thicknesses of up to 0.7 mm is used in specific embodiments. In an alternative, plasma activation and welding of PEEK PAKS of greater PEEK thickness enable welding of materials having a greater thickness.

### Lap Joint Sample Preparation

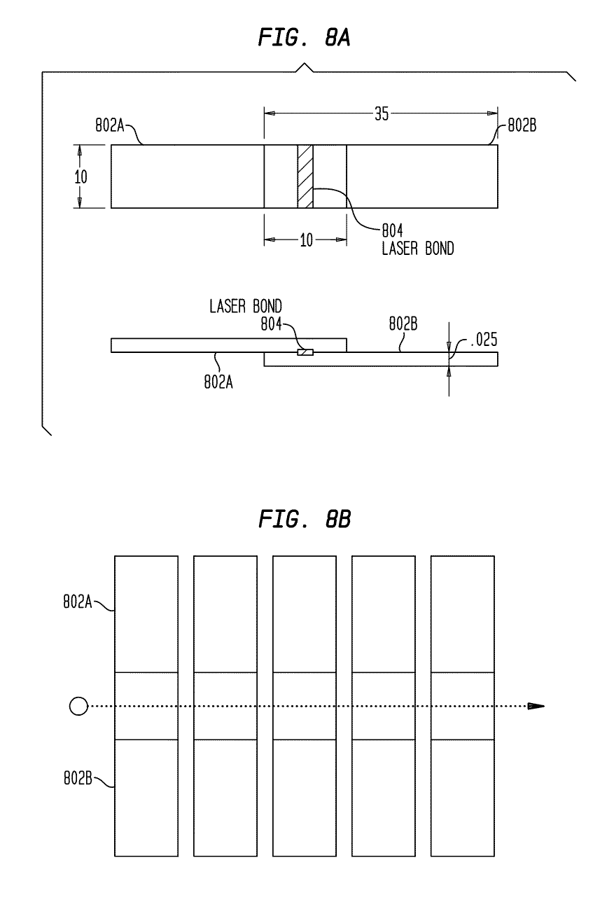

In this embodiment, rectangular strips measuring 35 mm by 10 mm are cut from the 250a and 250c PEEK films using conventional scissors. Ethanol (70%) was used to wipe the surface of the bond interface clean. Clearweld is painted evenly onto one of the contact surfaces only (interface of bottom layer). Five samples at a time are welded. The strips 802 are laid out in the lap joint configuration with a 10 mm overlap, as shown in FIGS. 8a and 8b. A glass plate (10 mm thick) was used to cover the strips and provide the necessary pressure to form intimate contact at the bond interface. Visual inspection for visible damage such as burning of the bonds is made using an optical microscope (Leica Microsystems Inc.).

### Transmission Laser Welding

A pulsed fiber laser (LF200, Alltec, Selmsdorf, Germany) is used with a wavelength of 1060 nm and maximum power of 20 W. For all welded samples, the maximum pulse frequency of the laser (80 kHz) is selected. For this example, the focal length and working distance of the laser are 165 mm and 200 mm, respectively.

Two laser parameters are varied: laser intensity and path speed. Five focal plane speeds (4, 8, 16, 32, 64 mm/s) and two intensities (10 and 20 W) are investigated. This provided 10 combinations for each of the two PEEK morphologies; totalling 20 groups of n=5 per group (see Table 4). It should be noted that the speeds correspond to the speed of the laser beam at the focal plane and not the exact speed of the beam at the bond interface 804. The calculated actual beam scan speeds at the bond interface are 4, 10, 19, 39, and 77 mm/s.

TABLE 4
Sample details

| | | Sample Size | | Intensity | Speed |
|---|---|---|---|---|---|
| Group | Label | (n) | Morphology | (W) | (mm/s) |
| 1 | 1004a | 5 | amorphous | 10 | 4 |
| 2 | 1008a | 5 | amorphous | 10 | 8 |
| 3 | 1016a | 5 | amorphous | 10 | 16 |
| 4 | 1032a | 5 | amorphous | 10 | 32 |
| 5 | 1064a | 5 | amorphous | 10 | 64 |
| 6 | 2004a | 5 | amorphous | 20 | 4 |
| 7 | 2008a | 5 | amorphous | 20 | 8 |
| 8 | 2016a | 5 | amorphous | 20 | 16 |
| 9 | 2032a | 5 | amorphous | 20 | 32 |
| 10 | 2064a | 5 | amorphous | 20 | 64 |
| 11 | 1004c | 5 | semi crystalline | 10 | 4 |
| 12 | 1008c | 5 | semi crystalline | 10 | 8 |
| 13 | 1016c | 5 | semi crystalline | 10 | 16 |
| 14 | 1032c | 5 | semi crystalline | 10 | 32 |
| 15 | 1064c | 5 | semi crystalline | 10 | 64 |
| 16 | 2004c | 5 | semi crystalline | 20 | 4 |
| 17 | 2008c | 5 | semi crystalline | 20 | 8 |
| 18 | 2016c | 5 | semi crystalline | 20 | 16 |
| 19 | 2032c | 5 | semi crystalline | 20 | 32 |
| 20 | 2064c | 5 | semi crystalline | 20 | 64 |

The welded lap joint samples are tested in tension using an Instron 5543 (Instron Pty Ltd, Melbourne, Australia) with a 1 kN load cell using a cross-head displacement rate of 5 mm/min. Lap joint samples are mounted in the Instron grips with a 40 mm gauge length, and the bond line centered within the gauge length. Each sample is tested until failure and the load at failure (N) is recorded. The normalized lap-shear strength, LSS (MPa), is calculated as the force at failure, F_{f} (N), divided by the contact area, A_{c} (mm²). The contact area is assumed to be the area the laser covers at the interface site, which is 8 mm2, based on an estimated laser diameter of 0.8 mm at the bond interface and a bond length of 10 mm.

The mode of failure for each joint is identified by inspection based on the standard definitions described in Table 5, which have been adapted from classifications used with adhesive-based joints. There are two main types of failure: interfacial and substrate. Interfacial failure occurred when the joint failed immediately at the interface between the opposing surfaces. If the joint failed within the substrate and the bond is still intact, this is termed substrate failure. There are different categories of substrate failure, which are broadly classified as substrate type I and substrate type II failure. Type I failure is where the substrate yielded and failed away from the interface; this is actually a measurement of the properties of the substrate and is the most desired result. In type II failure the bond remained intact but the substrate failed proximal to the interfacial region. In this type of failure the measured failure level is not typical of the substrate material properties.

### Surface Characterization

Post-failure characterization of the bond interface is conducted using SEM. Samples are mounted on aluminum stubs using double sided conductive tape. The samples are sputter coated with gold using a sputter coater (K550X, Quorum Emitech, Kent, UK) at 25 mA for 2 min, which gave a 15 nm coating thickness. A Phillips XL30 SEM is used at an acceleration voltage of 15 kV.

Two representative welds are selected for analysis of the weld cross-section (post-mechanical testing). Samples that failed in the Substrate Type I mode (Table 5) are selected for analysis to ensure the weld interface is intact. The welded samples are cut along the centre of the weld in the direction of the laser path. The samples are embedded in epoxy and polished on a Struers polisher with progressively finer grades of grit paper. The cross-section of the two welds is viewed under an optical microscope and SEM.

### Statistics

The mean LSS and corresponding standard deviation (SD) are calculated for each group (n=5). A square-root transformation is applied to the data in order to apply parametric tests. This experiment is a 2×2×5 factorial (morphology x power x speed) with five replications of each factor level combination; totaling 100 samples. A three-way analysis of variance (ANOVA) is employed to determine whether the effects of the three variables (power, speed and morphology) are statistically significant (Minitab, Version 15.1). Statistical significance is set at p<0.05.

### Results

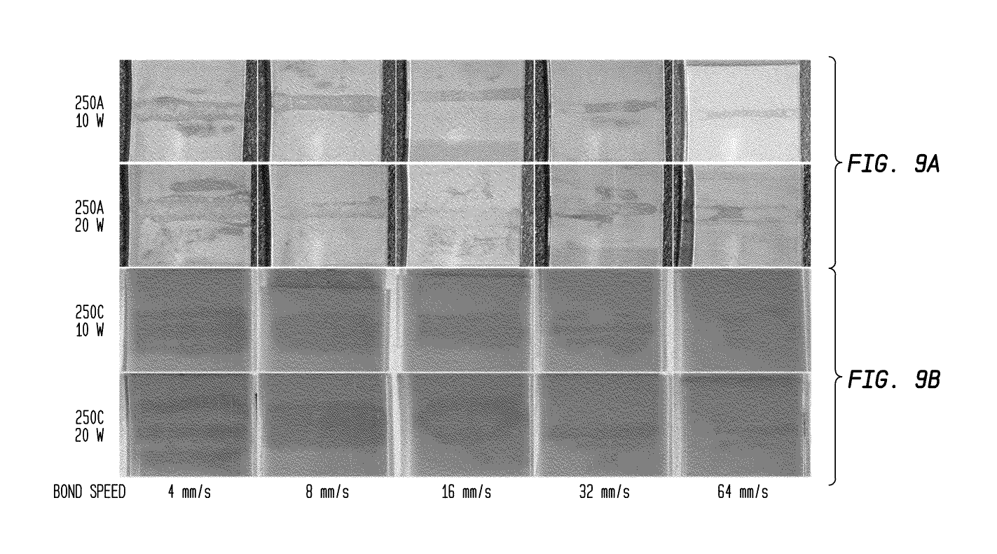

FIG. 9a includes representative optical micrographs of semi-crystalline joints before mechanical testing, while FIG. 9b includes representative amorphous micrographs of semi-crystalline joints before mechanical testing. As may be seen, the bond or weld line is thicker and darker in the groups that had the slower laser speed. The heat affected zone (HAZ) is clearly evident in the 4 mm/s and 8 mm/s groups. No HAZ could be seen at 64 mm/s speed, with the weld line appearing disjointed. Additionally, visual inspection after laser bonding showed that material damage in the form of burning occurred only in the 2004a group (amorphous; 20 W; 4 mm/s). Significant damage is seen in only one sample (see FIG. 10a), while the others displayed minor burning at the edges. No damage is noted in any of the semi-crystalline samples. Furthermore, the distinction between bond width and the heat affected zone (HAZ) is indicated in FIG. 10B. The HAZ is more prominent at slower speeds, while no HAZ is evident in the images for the faster speeds. The HAZ, which is outside the area exposed to the laser beam, is indicated.

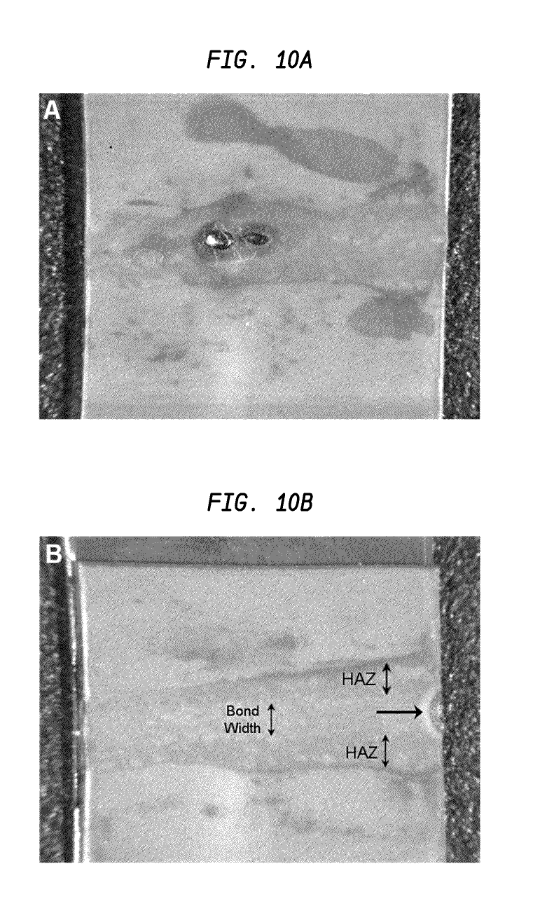

FIG. 11a is a graph illustrating mechanical testing results (mean lap-shear strength (LSS), MPa) for the amorphous morphology, while FIG. 11b illustrates mechanical testing result for the semi-crystalline (right) morphology. The illustrated error bars indicate standard deviation (SD). The LSS is seen to increase with a reduction in the scan speed. The greatest LSS is measured in the semi-crystalline PEEK sample bonds at 4 mm/s and 8 mm/s for both 10 W and 20 W laser power levels (ranging from 22.2 to 25.2 MPa). The LSS for all the semi-crystalline bonds are greater than for the amorphous PEEK sample bonds, for all power and speed settings (p<0.05). Statistical analysis indicated that there is no statistical difference in LSS results between the two laser intensities (p=0.325).

The failure load is recorded as the load at which the joint broke into two separate pieces; in this example this occurred with bonds that failed with interfacial and mixed failure modes (Table 5). The samples that failed with Substrate Type I mode did not break into two separate pieces, but failed in the typical tensile manner of PEEK, in which the substrate yielded and then deformed plastically with corresponding necking; typical of ductile polymers. In the cases where the failure mode is Substrate Type I, the yield load is taken as the failure load. This Substrate Type I failure mode occurred in four out of five samples for group 1008a (amorphous; 10 W; 8 mm/s), indicating that the bond is stronger than the substrate material. Overall, in the amorphous group, 88% failed interfacially, while in the semi-crystalline group only 38% failed interfacially; the rest of the bonds failed in a mixed or substrate manner. Furthermore, in the semi-crystalline group, interfacial failure only occurred in bonds with tracking speeds of 32 mm/s and 64 mm/s, no interfacial failure is seen for any of the lower tracking speeds. For the amorphous bonds, it is the 1008a group that had the majority of the Substrate Type I failures (4 out of 5), whereas only one Substrate Type I failure occurred in the 1004a (amorphous; 10 W; 4 mm/s) and 2004a (amorphous; 20 W; 4 mm/s) groups each.

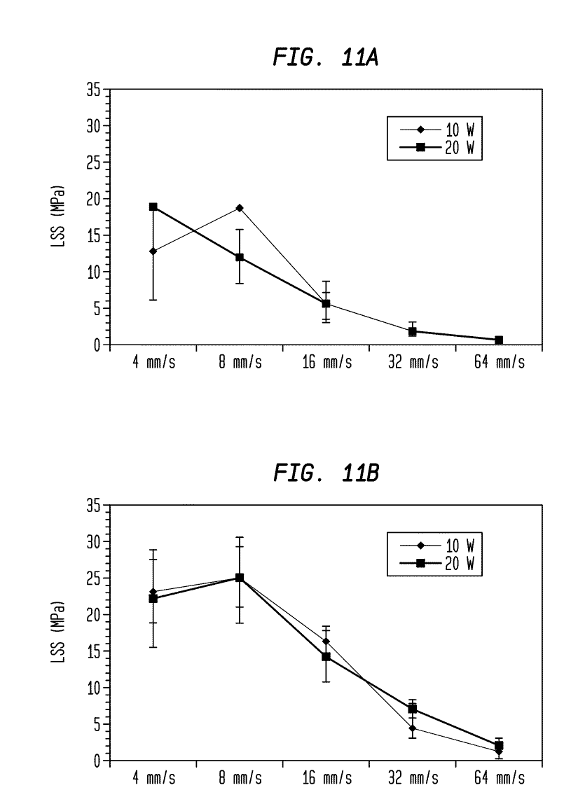

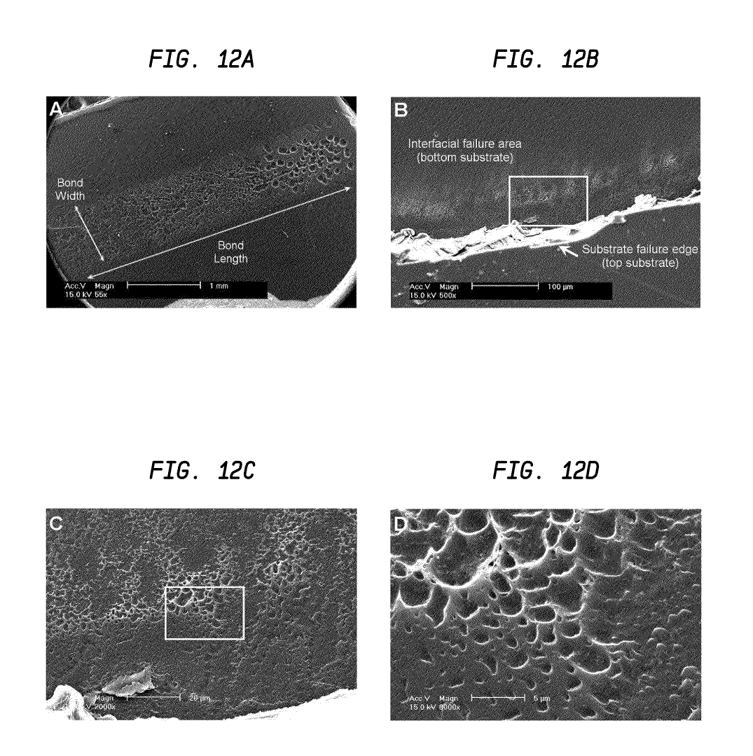

FIGS. 12a-12d show post-failure SEM images of the bond interface for two samples which are representative of typical failures for the two polymer morphologies (1016a: amorphous; 10 W; 11 mm/s, and 2008c: semi-crystalline; 20 W; 8 mm/s). FIG. 12a shows the surface of an interfacially failed sample from the amorphous group (1016a), with the weld width and length indicated. Laser induced pores are clearly evident across the weld line, and a significant number of relatively large pores are also present. FIGS. 12b and 12c are images of a semi-crystalline sample that failed in a Mixed Type I mode (2008c). The interfacial and substrate failure areas are marked. Within the interfacial failure zone at higher magnification, the pores are plastically deformed in the direction of tension (see FIG. 12d), indicating some substrate failure within the interfacial failure section. This deformation is not seen at the same magnification in the sample of FIG. 12a, which presents purely interfacial failure.

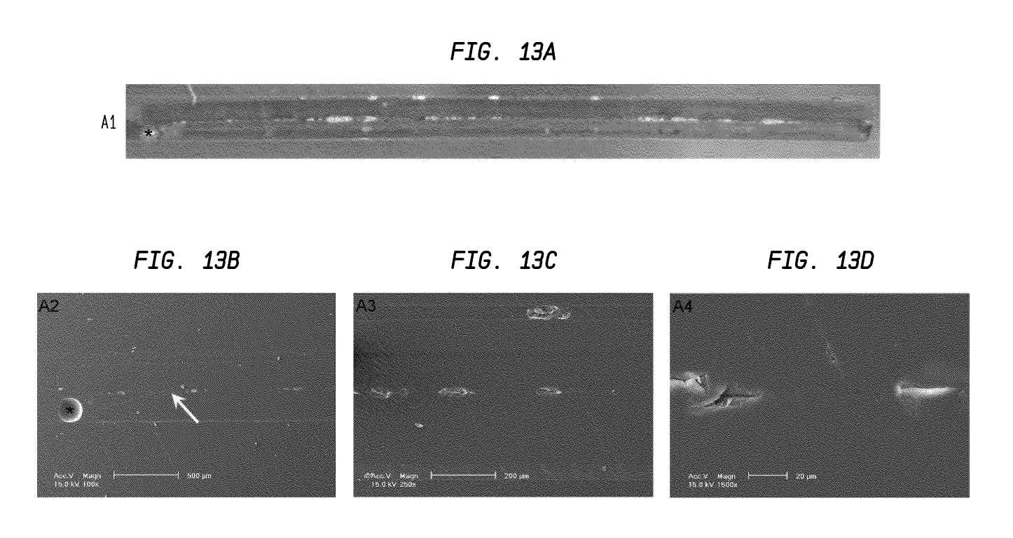

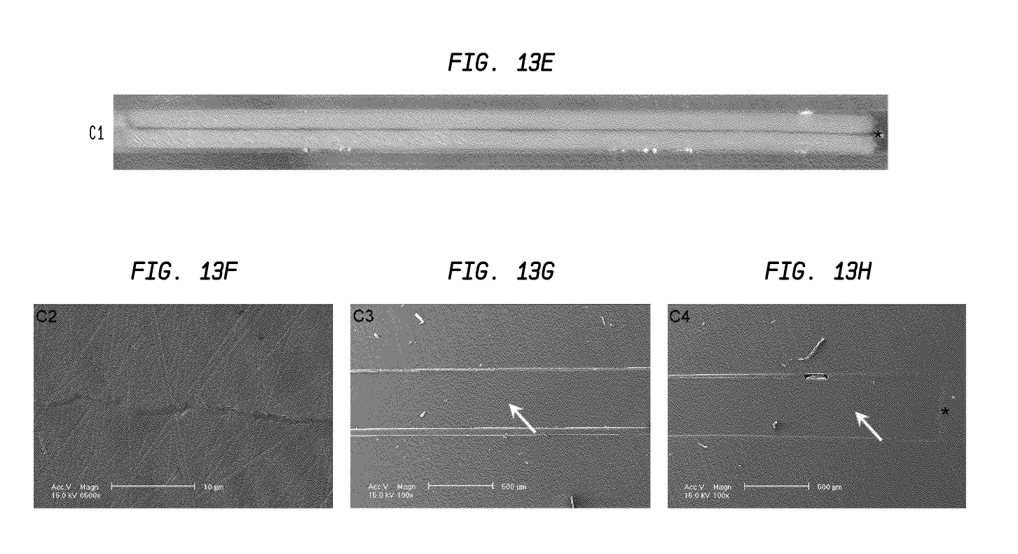

FIG. 13 shows optical micrographs and corresponding SEM images of two representative cross-sections; the amorphous weld (1004a) and the semi-crystalline weld (2008c). A1 and C1 are optical microscope images of an amorphous weld (1004a) and a semi-crystalline weld (2008c), respectively. A2 to A4 and C2 to C4 are the corresponding SEM images. The asterisks in A2 and C4 indicate the positions of the corresponding optical microscope images A1 and C1. The arrow in A2 indicates the weld line for the amorphous sample. The arrows in C3 and C4 indicate the approximate location of the weld line for the semi-crystalline sample.

### Discussion

The results indicated that the laser power did not affect the bond strength, for the two representative laser power settings assessed. However, burning occurred at the higher power and at the slowest speed, where the laser beam interacted with the material for the longest time. In similar studies, Van de Ven et al. found the highest laser power (19 W) and lowest scan speed (40 mm/s) that they investigated resulted in thermal decomposition when welding polyvinylchloride (PVC) T-joints using an 808 nm diode laser. Thin and weak welds formed at a lower laser power (16 W) and a faster scan speed (70 mm/s). They found that optimal bond strength is achieved at a power that raised the temperature at the interface to just below the temperature where PVC decomposition occurs. Potente et al. examined PEEK T-joint specimen geometries fabricated using a solid-state laser with wavelength 1064 nm and maximum power of 150 W. The transparent layer is 4 mm thick (compared to 0.25 mm in the present study). A number of process parameters are assessed for their influence on welding strength (including pre-drying PEEK prior to welding). They also found that weld strength increased with rising laser intensity at the bond interface in conjunction with falling scan speeds; a maximum weld strength of 40 MPa is achieved.

The strongest bonds for each morphology group are achieved with the two lowest speeds (4 mm/s and 8 mm/s). Slower speeds have also resulted in stronger bonds in other polymer laser welding studies. Mian et al. assessed two scan speeds when welding polyimide to titanium in a lap joint configuration, 100 mm/min and 1300 mm/min, and found that the slower scan rate resulted in a 40% strength increase. The group attributed this to the slower speed allowing more time for the laser to interact with the material, and therefore increasing the degree of melting and thus fusion. As mentioned earlier, Potente et al. also found that slower scan rates for quasi-simultaneous laser welding resulted in stronger bonds, where the maximum strength is achieved at the slowest scan rate of 100 mm/s.

The HAZ is well defined in the welding of metals. It is the region of material adjacent to the weld where microstructural and property changes occur as a result of heat conduction from the weld site. A HAZ can also occur in polymer welding. Newaz et al. noted a HAZ in the polyimide in their study; the bond width is evident, and a HAZ is present on either side of the bond, as indicated by bubbles. FIGS. 9 and 10 indicate the presence of a HAZ in the present study. Certain changes in polymer structure can be detrimental to the strength of the joint, as seen in the study by Cakmak et al. where failure occurred directly through the HAZ in vibration welded PEN butt joints. This is the result of the orientation of the naphthalene crystal plane within the HAZ, which is parallel to the weld line, with weak interchain bonds between planes.

The limitations of this example include the accurate estimation of LSS area, the application of Clearweld and the uniformity of pressure applied to the samples for welding. Calculation of LSS requires the exact A_{c} of the weld to be known. This area is assumed to be the area covered by the laser beam during the welding process, which is based on the estimated laser diameter at the interface. This is considered to be the theoretical A_{c}, however as FIG. 9 shows, the actual A_{c} varied between groups. Samples that are laser scanned at 4 mm/s appear to have a larger A_{c} than the laser beam, whereas the 64 mm/s bonds appear only partially bonded. This difference is proposed to be due to more extensive melting (HAZ) at the slower speed from broader heat transmission, but on the other hand, it is not established whether the interface within the HAZ actually bonded. For this reason we believe that calculation of the LSS based on the appearance of the bond area may be misleading, since the real bonding efficiency of the laser may still not be taken into account. The bond efficiency is defined herein as the actual A_{c} divided by the theoretical Ac.

The amount of Clearweld applied to the interface surface may be an important factor in the weld process. Consistency when applying the coating is attempted, through use of one operator for application of the Clearweld, however the variability inherent in manual processing is noted. The generation of heat within the joint is dependent on the amount of absorbent applied, which has been noted to affect the quality of the bond. A number of methods for controlled delivery of surface treatment are commercially available, such as ink jet printing and spray/needle dispensing.

The pressure applied by the glass plate to the lap joint samples may not have been evenly distributed during the laser welding process. Pressure in the weld zone is a vital parameter in laser welding; it provides the intimate contact required for thermal conduction between the absorptive and transmissive parts. Without adequate pressure, gaps may be present and may not bridge during the welding process. This pressure may also promote squeeze flow of the molten plastic, improving mechanical strength. However, Potente et al. found that clamping pressure did not significantly influence the resultant bond strength of PEEK. They assessed clamping pressures between 1 MPa and 3 MPa and found the highest weld strength occurred at 1.5 MPa, but is not statistically significant. Van de Ven et al. assessed the influence of clamping pressure (between 0.5 MPa and 4 MPa) for laser welding of PVC. Although the differences they found are not significant, they concluded from trends in the data that the highest quality welds occurred at 2.5 MPa clamp pressure, with a decline in quality below and above this value. With respect to this example, inconsistent weld lines seen in joints created with the faster speeds (FIG. 9) may have been the result of gaps present at the interface prior to welding.

The biocompatibility of Clearweld, and other utilized laser absorbent material additives, needs to be considered for application in medical devices. There are no publications indicating that Clearweld has been tested in a medical device, as it is a relatively new product to the market. For non-implantable applications, Clearweld has been tested for biocompatibility and cytotoxicity, and meets USP Class VI requirements.

### Conclusion

This example assessed the efficacy of laser welding of PEEK using Clearweld as the infrared absorbing medium. Three variables are investigated: the laser power (10 W or 20 W), the path speed (4, 8, 16, 32 or 64 mm/s), and the morphology (amorphous or semi-crystalline). Both amorphous and semi-crystalline PEEK film are successfully laser welded using Clearweld; the bonds formed with the semi-crystalline material are stronger than those for the amorphous material.

The following conclusions can be made from the results:
Maximum weld strength is achieved with the slowest laser speeds (4 mm/s and 8 mm/s).
There is no difference in strength between the two laser powers assessed (10 W and 20 W).
Amorphous PEEK is susceptible to heat damage at the higher power, while no visual evidence of heat damage is evident in semi-crystalline PEEK.
Semi-crystalline PEEK weld strengths are greater than the amorphous PEEK weld strengths, at all speeds and power settings.

The laser welding method using Clearweld to weld PEEK films shows suitability for applications in the medical device industry, particularly where strength is essential. However, as noted above, Lumogen may be used as an alternative to Clearweld and the description of Clearweld herein is not intended to limit embodiments of the present invention.

### Example 3

### Transmission Laser Welding (Quasi-Simultaneous Irradiation) of PEEK Plates

### Purpose

Example 3 illustrates embodiments of the present invention in which PEEK plates are welded using Lumogen as the absorbing pigment. A matrix of time (s) versus power (W) is used to determine optimal exposure time and laser power configurations with respect to bond strength.

### Background

This assessment utilized injection molded PEEK lap joint plates. The plate geometry is designed for optimal lap-shear mechanical testing, and each plate had three step thicknesses: 1.00 mm, 0.67 mm and 0.33 mm. PEEK plates are injection molded with or without Lumogen pigment.

A preliminary assessment is made using either Clearweld (applied to the interface between two unpigmented PEEK plates) or Lumogen pigmented plates. Bonding of all three step configurations is assessed (i.e., laser passing through 0.33 mm, 0.67 mm, and 1.00 mm thicknesses). To provide sufficient grip length for mechanical testing, without the need to cut the plates, the 0.33 mm step is welded to the 1.00 mm step and vice versa.

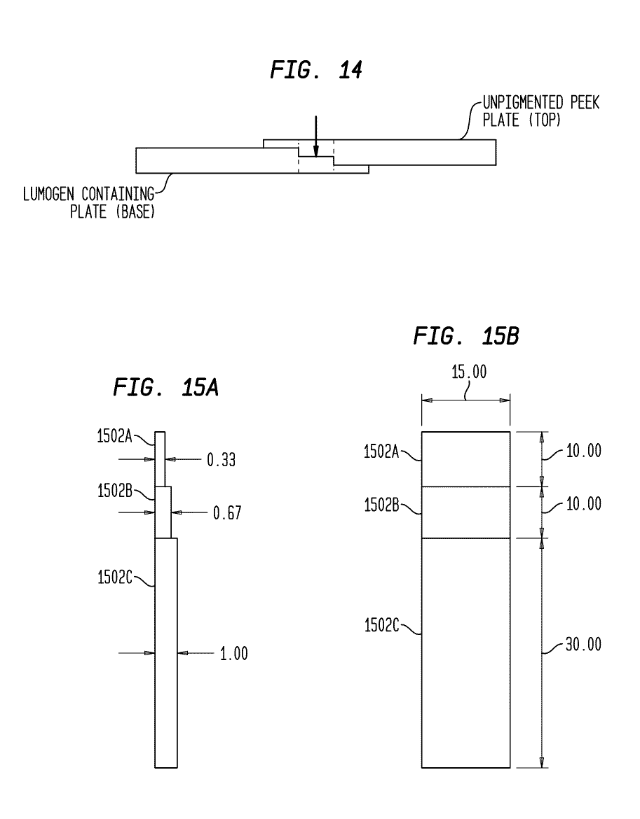

Both Clearweld and Lumogen bonds demonstrated similar strengths, however, the Clearweld bonds displayed highly non-uniform bonded regions and HAZs. Also, due to the asymmetry of the 0.33 to 1.00 mm joint and the 1.00 to 0.33 mm joint, the mechanical behavior of the test in tension is also asymmetrical and thus difficult to interpret. It is decided to conduct further work on the 0.67 to 0.67 mm step thickness configuration, as shown in FIG. 14. The arrow in FIG. 14 indicates the laser beam entering the top plate through the 0.67 mm thickness step.

### Study Design

Table 6 shows the matrix that is devised to determine the optimal parameter configurations for exposure time (s) and laser power (W). For each configuration, five samples are welded (n=5). A cross in the matrix indicates where plates are not welded for a given configuration either due to known burning or non-bonding of the sample.

TABLE 6
Matrix for parameter optimisation

| | | | | | | |
|---|---|---|---|---|---|---|
| | 40 W | 50 W | 60 W | 70 W | 80 W | |
| 50 s | n = 5 | n = 5 | n = 5 | n = 5 | X | 50 s |
| 40 s | X | n = 5 | n = 5 | n = 5 | X | 40 s |
| 30 s | n = 5 | n = 5 | n = 5 | n = 5 | n = 2 | 30 s |
| 20 s | X | n = 5 | n = 5 | n = 5 | X | 20 s |
| 10 s | X | X | n = 6 | n = 5 | n = 5 | 10 s |
| | 40 W | 50 W | 60 W | 70 W | 80 W | |

### Materials

FIGS. 15a and 15b are side and from views of PEEK lap joint plates 1502 with dimensions as shown. The plates are injection molded from PEEK pellets obtained from Victrex. For the plates containing Lumogen, pellets pigmented with Lumogen are added to the molding barrel to create a final Lumogen content of 5 Oppm in the plates.

### Sample Preparation

The plates are welded in the configuration shown in FIG. 14. The Lumogen plate is placed at the base, and the unpigmented plate on top. A glass plate is placed on top of the sample to provide sufficient weight for intimate contact.

### Transmission Laser Welding

A GSI fibre laser with a wavelength of 1080 nm and a continuous wave profile is used to bond the samples. The power range for the laser is 5 to 100 W. A focal length and a working distance of 224.5 mm and 240 mm, respectively, are used. At this working distance, the estimated laser diameter is 500 microns.

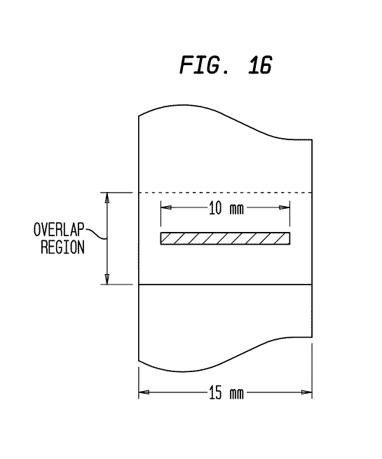

To create a quasi-simultaneous laser beam the maximum scan rate (10,000 mm/s) for the galvanometric head is employed. The scanning distance for the weld is 10 mm, as shown in FIG. 16. As shown, the quasi-simultaneous exposure length is 10 mm centered on the width of the 15 mm wide plates.

### Mechanical Testing

The lap joint samples are tested in tension using an Instron 5543 and a 1 kN load cell. A cross-head displacement rate of 2 mm/min is used. Samples are mounted in the Instron grips with a 40 mm gauge length, and the bond line centered within the gauge length. Each sample is tested until failure. The load (N) and corresponding cross-head displacement (mm) are recorded. The LSS (MPa), or bond strength, is calculated as Ff (N) divided by A_{c} (mm²). A_{c} is assumed to be the area the laser covered at the interface site, which is 5 mm², based on an estimated laser diameter of 0.5 mm at the bond interface and a bond length of 10 mm. The mode of failure for each joint is identified with the naked eye.

### Results

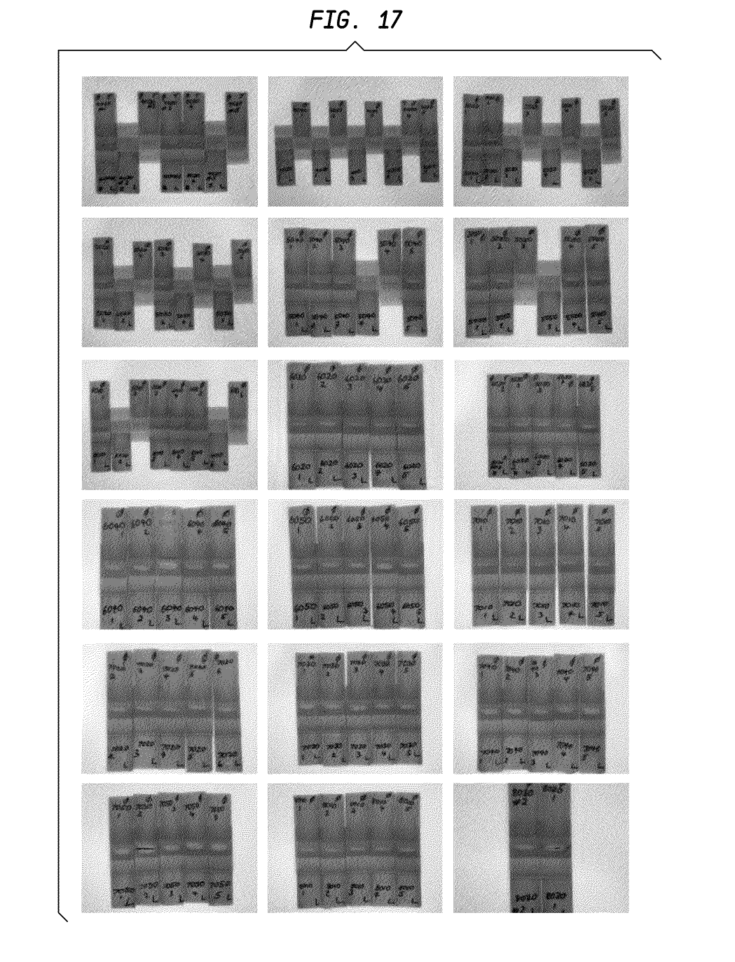

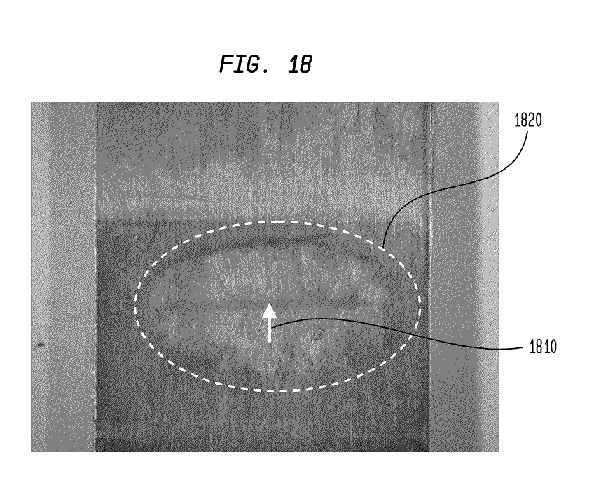

Images of the intact welds are shown in FIG. 17. Due to the relatively long exposure time of the samples to the laser (10 to 50 s), crystallization of the weld region occurred in all plates, with a clearly identifiable HAZ as shown in FIG. 18. More particularly, FIG. 18 shows the underside (Lumogen containing plate) of sample 7050_{—5 (}70 W and 50 s). The arrow 1810 indicates the weld line seen as the dark straight horizontal line. The HAZ is circled by the dotted line 1820, and the boundary of the HAZ is visible within the dotted line. The HAZ is always more prominent on the Lumogen plate side of the weld compared with the unpigmented plate side.

The crystallization appeared as an opaque color change around the weld region. All groups displayed a change in color indicating crystallization, however, the groups within the top right corner of the matrix (longer time and higher power) displayed increased crystallization (as determined by the extent of the color change).

Although five samples per group are welded, not every sample bonded (particularly for the lower power groups). The number of samples that resulted in a bond within a group is recorded (Table 7). Although some of the samples physically bonded at the time of welding, they were relatively weak such that they failed either during transportation or during placement in the Instron for testing.

TABLE 7
Bonding efficiency

| | | | | | | |
|---|---|---|---|---|---|---|
| | 40 W | 50 W | 60 W | 70 W | 80 W | |
| 50 s | n = 5 | n = 5 | n = 5 | n = 5 | X | 50 s |
| | n_{b} = 2 | n_{b} = 4 | n_{b} = 5 | n_{b} = 5 | | |
| | nₘ = 1 | nₘ = 4 | nₘ = 5 | nₘ = 5 | | |
| 40 s | X | n = 5 | n = 5 | n = 5 | X | 40 s |
| | | n_{b} = 5 | n_{b} = 5 | n_{b} = 5 | | |
| | | nₘ = 3 | nₘ = 5 | nₘ = 5 | | |
| 30 s | n = 5 | n = 5 | n = 5 | n = 5 | n = 2 | 30 s |
| | n_{b} = 3 | n_{b} = 4 | n_{b} = 5 | n_{b} = 5 | n_{b} = 2 | |
| | nₘ = 1 | nₘ = 0 | nₘ = 5 | nₘ = 5 | nₘ = 2 | |
| 20 s | X | n = 5 | n = 5 | n = 5 | X | 20 s |
| | | n_{b} = 2 | n_{b} = 5 | n_{b} = 5 | | |
| | | nₘ = 2 | nₘ = 5 | nₘ = 5 | | |
| 10 s | X | X | n = 6 | n = 5 | n = 5 | 10 s |
| | | | n_{b} = -4 | n_{b} = 5 | n_{b} = 5 | |
| | | | nₘ = 4 | nₘ = 5 | nₘ = 5 | |
| | 40 W | 50 W | 60 W | 70 W | 80 W | |

n: Number of samples welded in group
n_{b}: Number of samples that resulted in a bond at the time of welding
nₘ: Number of samples that remained intact for mechanical testing

Table 8 shows the mean LSS in MPa for each group in the matrix. Also indicated is the SD. The highest strengths occurred in the top right region of the matrix (higher power and longer time). The potential for burning however is increased in this region.

TABLE 8
Mean LSS (MPa) and SD

| | | | | | | |
|---|---|---|---|---|---|---|
| | 40 W | 50 W | 60 W | 70 W | 80 W | |
| 50 s | 5 | 8 (±3) | 30 (±16) | 53 (±12) | X | 50 s |
| | MPa | MPa | MPa | MPa | | |
| | n = 1 | n = 4 | n = 5 | n = 5 | | |
| 40 s | X | 7 (±3) | 27 (±7) | 37 (±19) | X | 40 s |
| | | MPa | MPa | MPa | | |
| | | n = 3 | n = 5 | n = 5 | | |
| 30 s | 3 | 0 | 13 (±13) | 35 (±7) | 62 (±9) | 30 s |
| | MPa | MPa | MPa | MPa | MPa | |
| | n = 1 | n = 0 | n = 5 | n = 5 | n = 2 | |
| 20 s | | 5 (±2) | 14 (±5) | 35 (±5) | X | 20 s |
| | | MPa | MPa | MPa | | |
| | | n = 2 | n = 5 | n = 5 | | |
| 10 s | X | X | 8 (±1) | 24 (±4) | 40 (±14) | 10 s |
| | | | MPa | MPa | MPa | |
| | | | n = 4 | n = 5 | n = 5 | |
| | 40 W | 50 W | 60 W | 70 W | 80 W | |

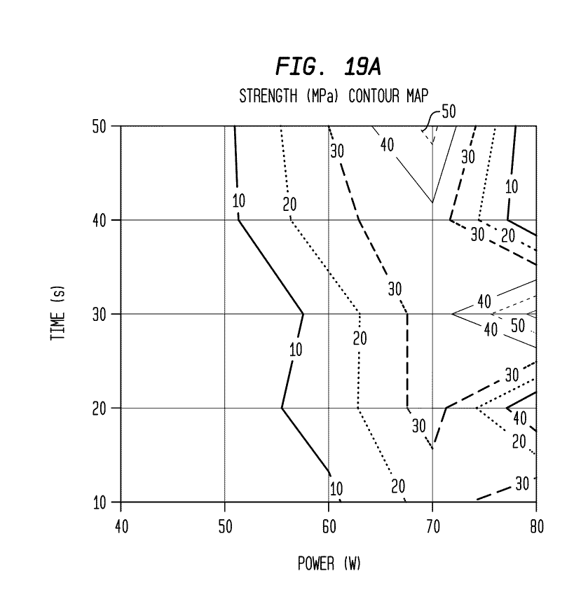

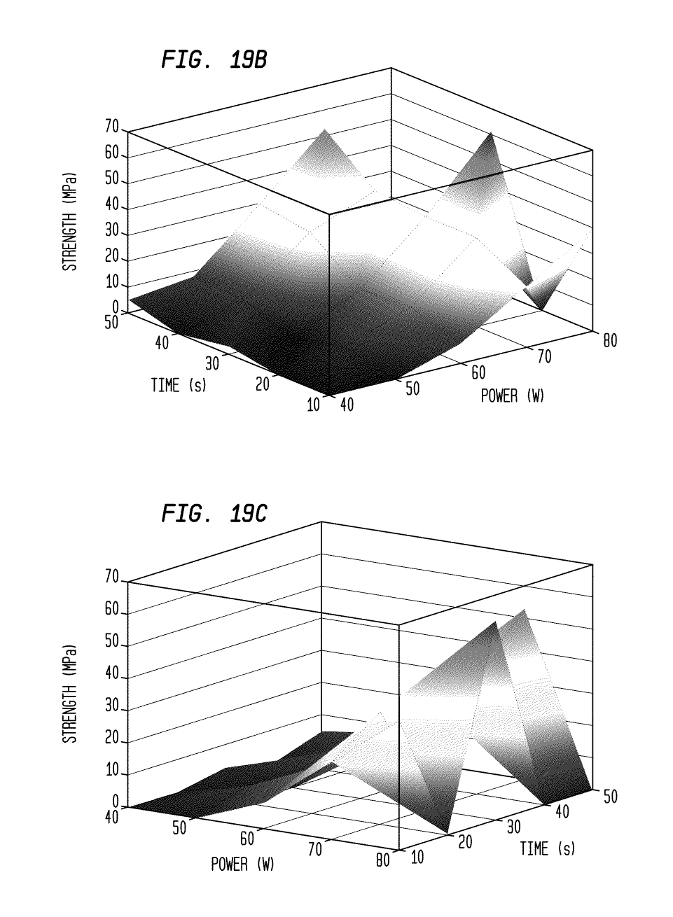

FIG. 19a shows the LSS results as a contour map. FIGS. 19b and 19c show the results as 3D surface plots, each from different angles, for improved visualization of trends. Table 9 indicates the failure modes and extent of burning (if any) for each sample in each group.

TABLE 9
Failure modes and extent of burning

| | | | | | | |
|---|---|---|---|---|---|---|
| | 40 W | 50 W | 60 W | 70 W | 80 W | |
| 50 s | I: 5/5 | I: 5/5 | M2: 4/5 | S2: 1/5 (B!) | X | 50 s |
| | | | M2/I: 1/5 | M2: 3/5 | | |
| | | | | M1: 1/5 | | |
| 40 s | X | I: 5/5 | M2/I: 5/5 | M2: 5/5 | X | 40 s |
| | | | | DB: 2/5 | | |
| 30 s | I: 5/5 | I: 5/5 | I: 4/5 | M2: 5/5 | S2: 1/2 (B!) | 30 s |
| | | | M2: 1/5 | DB: 1/5 | M2: 1/2 (B) | |
| 20 s | X | I: 5/5 | I: 5/5 | M2: 5/5 | X | 20 s |
| | | | | B: 1/5 | | |
| 10 s | X | X | I: 6/6 | I: 4/5 | M2: 5/5 | 10 s |
| | | | | M2: 1/5 | | |
| | 40 W | 50 W | 60 W | 70 W | 80 W | |

I: Interfacial failure
M1: Mixed Type 1 failure
M2: Mixed Type 2 failure
S2: Substrate Type 2 failure
B!: Extensive burn
B: Burn
DB: Dot burn

### Discussion

It would be expected that higher power and shorter exposure time would correlate with lower power and longer exposure time with regard to bond strength. This is not entirely the case for the matrix. The 40 W and 50 W power levels did not produce strong bonds regardless of laser exposure time. It is possible that these powers are not sufficient to efficiently penetrate the top plate and reach the Lumogen surface of the base plate. It is understood from power meter measurements of the transmitted laser through amorphous PEEK films, that crystallization decreases laser transmission, until a plateau is reached when maximum crystallization has occurred. The current results indicate that the plates undergo crystallization around the bond area during bonding. This may have been a contributing factor for the lack of bond strength in the lower powers. As the samples crystallized, the laser transmission also decreased, resulting in even less power reaching the interface.

The 80 W power level is the most volatile in terms of burning, and required monitoring throughout the welding time to ensure burning did not occur. It should be noted that the two samples of the 8030 group (80 W and 30 s) are not welded for the entire 30 s. The first sample began visible burning after approximately 19 s and the laser is immediately switched off. Extensive burning occurred along the weld line. The second sample that is welded began burning at 13 s (at which time the laser is immediately switched off). For both these samples however, a solid bond is formed and high strength values resulted.

The variability in the measured bond strengths within a group is relatively high for some groups. This could be due to a number of reasons, such as inhomogeneous Lumogen distribution, lack of intimate contact, or inadequate pressure.

For optimal bonding, the interface needs to reach a molten state. Assessment of the lap-shear failure modes suggested that the Interfacial failure types did not appear to have reached a molten state, whereas the Mixed and Substrate failure types showed indications of melting and fusion.

### Conclusions

The following conclusions can be made from the results of this example:
Higher power and longer exposure time resulted in the strongest bonds, but also increased the potential for burning.
Crystallization occurred around the weld region due to heat induced from the laser energy and extended exposure time.
The low laser powers (40 and 50 W) produced insignificant bond strengths, and the post-failure analysis indicated that the interface did not appear to reach a molten state.

### Example 4

### Protocol for Measuring Bond Strength of Polymer Films

### Purpose

As noted above, embodiments of the present invention facilitate bonding between polymeric surfaces. Example 4 demonstrates preparation of polymer films to measure the bond strength of various joining techniques. To measure bond strength, lap-shear joint configurations are constructed and tested in tension.

### Materials

This protocol has been prepared for measuring the bond strength of polymer films. The films vary in morphology (semi-crystalline or amorphous) and thickness (25 microns to 500 microns). For mounting of the tensile samples, Loctite 401 is to be used (or equally strong glue).

### Specimen Preparation

The films are bonded using either direct, adhesive or laser bonding.

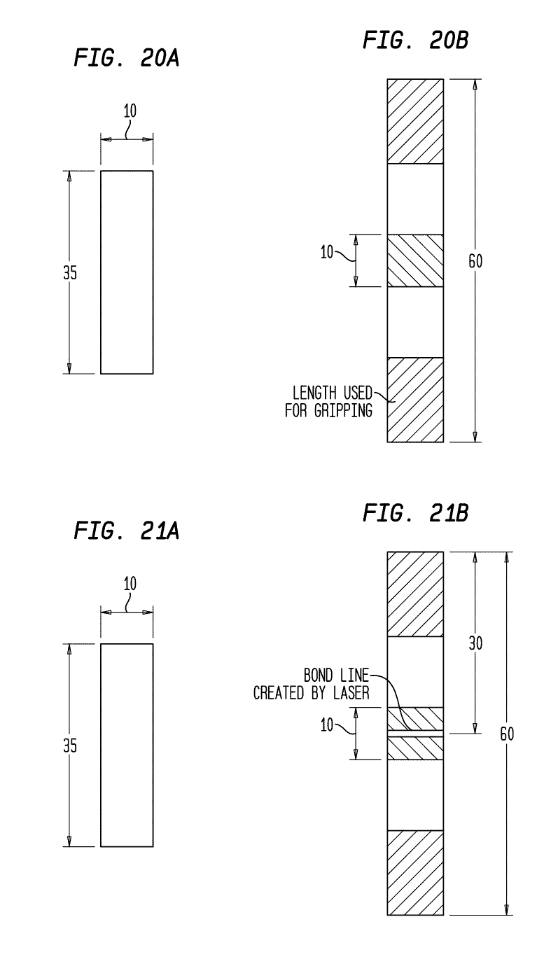

For direct and adhesive bonding methods, identical dimensions of the film are cut and bonded according to FIGS. 20a and 20b. The film thicknesses will vary depending on which film is utilized.

For the laser bonded samples, the same size individual film dimensions as in FIG. 20a are used. The films will overlap by 10 mm as well, shown in FIG. 20b, so the bond a 1 mm wide strip of 10 mm length, leaving a total bond area of 10 mm². Lengths of 10 mm at each end of the lap-shear configuration will be utilized for gripping into the Instron. The bond area will be a single laser line which has an approximate width of 1 mm. The configuration is shown in FIGS. 21b.

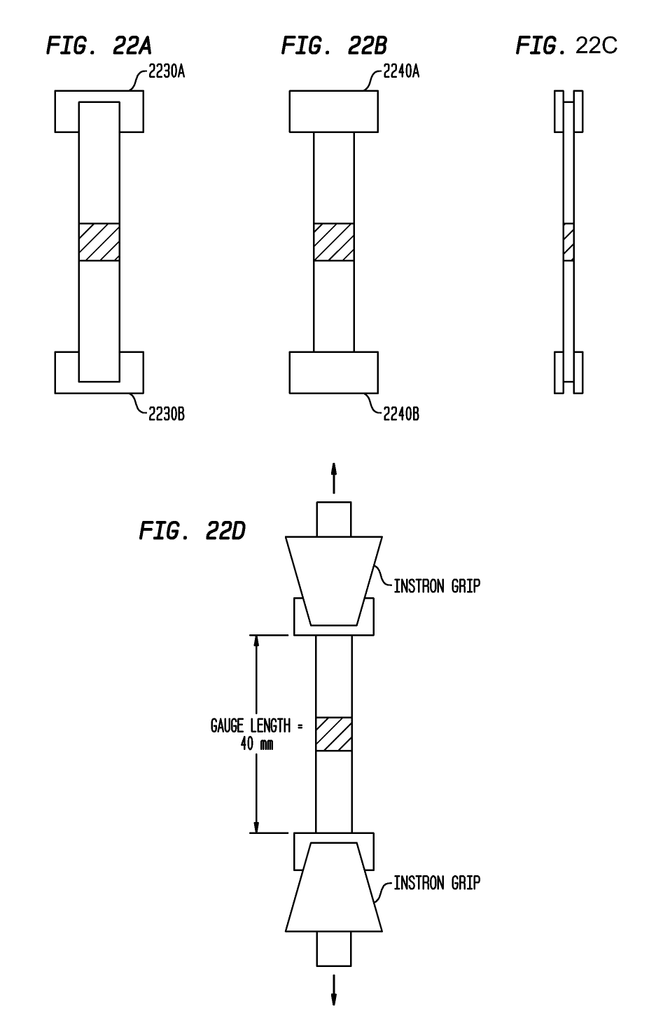

For tensile testing, if required for improved gripping, the ends of the bonded lap-shear samples are to be mounted onto cardboard. Business card paper can be used as the gripping material. Pieces 15 mm by 10 mm shall be cut and four per sample used. Loctite 401 (Henkel) adhesive (or equally strong glue) is to be used to bond the polymer films to the cardboard. FIGS. 22a illustrates the under layer of cardboard 2230 adhered using Loctite, while FIG. 22b illustrates the top layer of cardboard 2240 adhered using Loctite. FIG. 22c is a side view of the arrangement of FIGS. 22a and 22b. The arrangement of FIGS. 22a-22c creates typical bow-tie like configuration used in tensile testing. Emery paper can also be used between the grip and sample interface to improve gripping. The thicker ends allow for easier gripping into the Instron grips FIG. 22d illustrate the arrangement of FIGS. 22a- 22c is mounted in Instron grips where tension is then applied.

### Mechanical Testing

An Instron materials tester is to be used to test the lap-shear joints in tension. A 1 kN load cell is to be used for the tests. The cross-head speed is to be set at the chosen speed for the particular study (2 mm/min or 5 mm/min). The films shall be tested in tension until failure.
The load (N) and corresponding cross-head displacement (mm) should be recorded by the Merlin software (Instron).

### Documentation

For each sample a plot of the load versus extension will be made. The LSS (or bond strength), 6ₛ (Pa), is calculated as Ff (N) divided by A_{c} (mm²). The contact area is the bond area which is 100 mm² for the direct and adhesive bonds, and 10 mm² for the laser bonds.

σₛ=F_{f}/A_{c}

For groups with multiple samples, the mean LSS (σₛ_{_{—}}ₘₑₐₙ) and corresponding SD will be calculated per group. The mode of failure for each joint is identified using the definitions shown in Table 5. As such, tables of results, such as shown in Tables 10 and 11, may be generated to list the results of each sample.

TABLE 10
Format of sample results

| | | | Failure | | |
|---|---|---|---|---|---|
| Sample | | | load | LSS | |
| ID | Group | Details | (Ff, N) | (óₛ, Pa) | Failure mode |
| Number | Adhesive, | Comments | Value | Value | Classifications |
| | Direct or | | | | according to |
| | Laser | | | | Table 5 |

TABLE 11
Format of group results

| | Mean failure load | |
|---|---|---|
| Group | (F_{f}_{_{—}}ₘₑₐₙ, N) | Mean LSS (σ_{smean,} Pa) |
| Adhesive | Value (SD) | Value (SD) |
| Direct | Value (SD) | Value (SD) |
| Laser | Value (SD) | Value (SD) |

Figures showing graphically the LSS comparisons may also be generated. Appropriate statistical analysis is to be conducted to identify significant differences between groups.

### Conclusion

This protocol will generate mechanical strength data regarding the various bonding techniques assessed. It will allow comparisons to be made between bonding techniques to determine which technique generates the strongest bond. Furthermore, conclusions can be made regarding the optimal bonding conditions of the technique that results in the strongest bond.

### Example 5

### Protocol for Measuring Hermeticity of PEEK Plates

### Purpose

In this embodiment, the hermeticity of three polymer joining methods for PEEK: laser welding, plasma activated direct bonding and adhesive bonding are evaluated. As would be appreciated, the evaluated hermeticity refers to the integrity of the welded joint, rather than the hermeticity of the encapsulation as a whole. This protocol describes a hermeticity test method that utilizes PEEK plates fabricated for lap-shear tests. The test is relatively simple, and does not require the development of a prototype of the package in order to make preliminary conclusions regarding hermeticity of the bond. Note that this protocol makes no assessments of the polymer's inherent permeability; it is a test that investigates hermeticity of the seal created using the three methods described.

### Study Design

Three specific types of bonding will be tested: adhesive, direct and laser bonding. Table 12 below lists the details of the bonding groups.

TABLE 12
Details of bonding groups

| Bond | ID | Details | Sample size |
|---|---|---|---|
| Adhesive | A | Loctite and/or Epotek adhesive used | 5 |
| Direct | D | Plasma activated direct bonding | 5 |
| | | with heat and pressure | |
| Laser | L | Laser welding | 5 |

### Materials

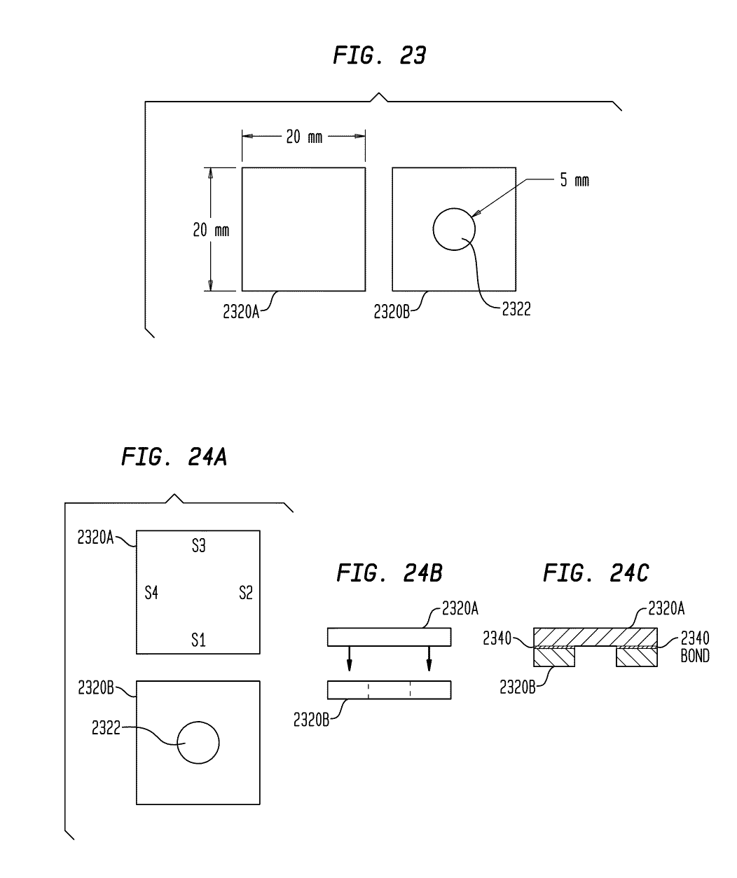

PEEK plates will be used to create a bonded open cavity. The injection molded plates for lap-shear bond tests will be utilized and cut into squares 2320 with a 20 mm edge length as shown in FIG. 23. The 1 mm thick step of the plate will be used, so all plates in this protocol are 1 mm thick. Half of the plates will require a hole 2322 of diameter 5 mm to be located at the center of the plate.

The adhesive used for the adhesive bonds will be Loctite 406 and/or Epotek 301-2.

Package design

FIGS. 24a-c shows the two plates 2322 from FIG. 23 in different arrangements. Specifically, FIG. 24b shows plates 2322 immediately prior to bonding and FIG. 24c shows the plates after bonding. A bond 2340 is formed between the plates using one of bonding methods A, D, or L noted above.

### Helium Leak Test

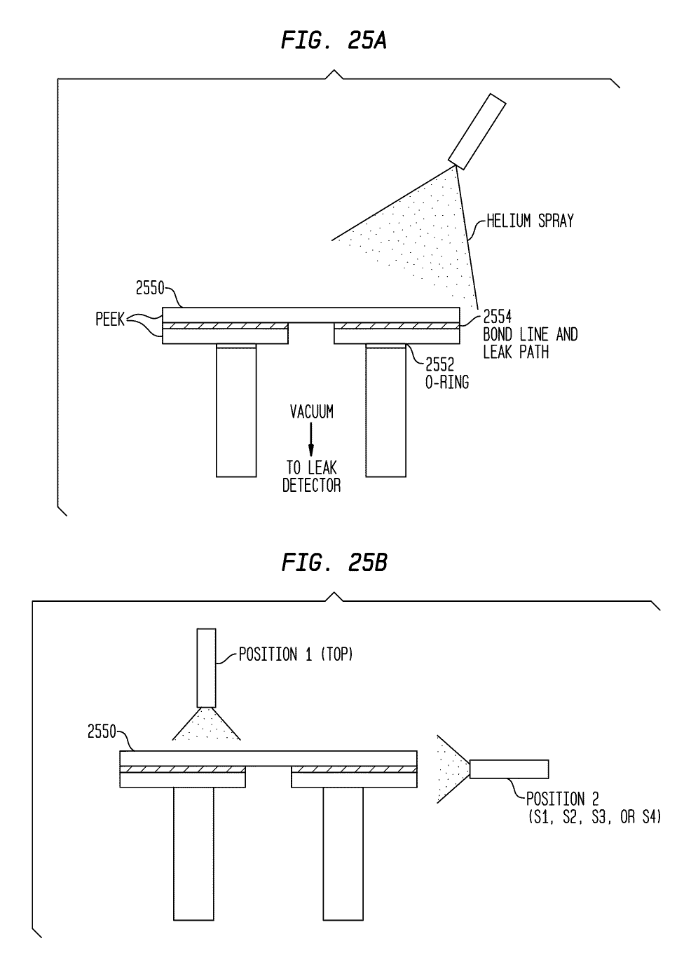

FIGS. 25a and 25b illustrates the set-up that may be used in accordance with embodiments of the present invention. The package 2550 will be placed on the O-ring 2552 with the hole-side down and inside the O-ring. A guide will be used to ensure the hole is placed within the O-ring. A vacuum seal will be created against the O-ring; it should be noted that it is essential that the O-ring seal is 100% hermetic. The only potential leak path is shown as 2254s in FIG. 25a. Helium gas is to be sprayed around the device using a spray nozzle.

Each package is to be tested using the spray positions shown in FIG. 25b. In position 1, the nozzle is held directly above the package and sprays continuously for 15 s. In position 2, the nozzle is held directly in-line with each of the four sides of the package, and sprays continuously for 15 s each.

### Documentation

This test is primarily qualitative. For each spray position, if no helium is detected, a PASS is to be given. If helium is detected, a FAIL is to be given for that position, and the reading recorded (if possible). The results are to be recorded in the format of Table 13 below.

TABLE 13
Format of sample results

| | Sam- | | | | | | Result |
|---|---|---|---|---|---|---|---|
| Bond | ple | Top | S1 | S2 | S3 | S4 | Summary |
| Adhesive | A1 | Pass | Pass | Fail | Pass | Pass | Fail |
| | | | | (1 × 10−5) | | | |
| Adhesive | A2 | Pass | Pass | Pass | Pass | Pass | Pass |

### Conclusion

is noted that the polymers are permeable to helium, hence this protocol will provide qualitative information regarding the hermeticity of three bonding techniques for PEEK: adhesive, direct or laser. This protocol does not investigate the permeability properties of the polymer itself.

### Example 6

### Protocol for Measuring Hermeticity of PEEK Capsules

### Purpose

In these embodiments, PEEK capsules created sealed using two polymer joining methods: laser welding and plasma activated direct bonding are evaluated.

### Background

Testing the hermeticity of a PEEK capsule involves assessing two potential ingress paths: ingress through the seal due to gaps at the bond interface, and ingress through the polymer bulk as a result of permeation. The protocol described in Example 5 addresses testing for leaks through the seal. This protocol focuses on testing the ingress of gas and vapor through the bulk material of the capsule and into the cavity. Surface treatment will be applied for the purpose of reducing the polymer's inherent permeability. This will therefore require tests to measure the permeability reduction of the surface treatments, as well as determining overall ingress into the bonded PEEK package.

### Permeability Reduction

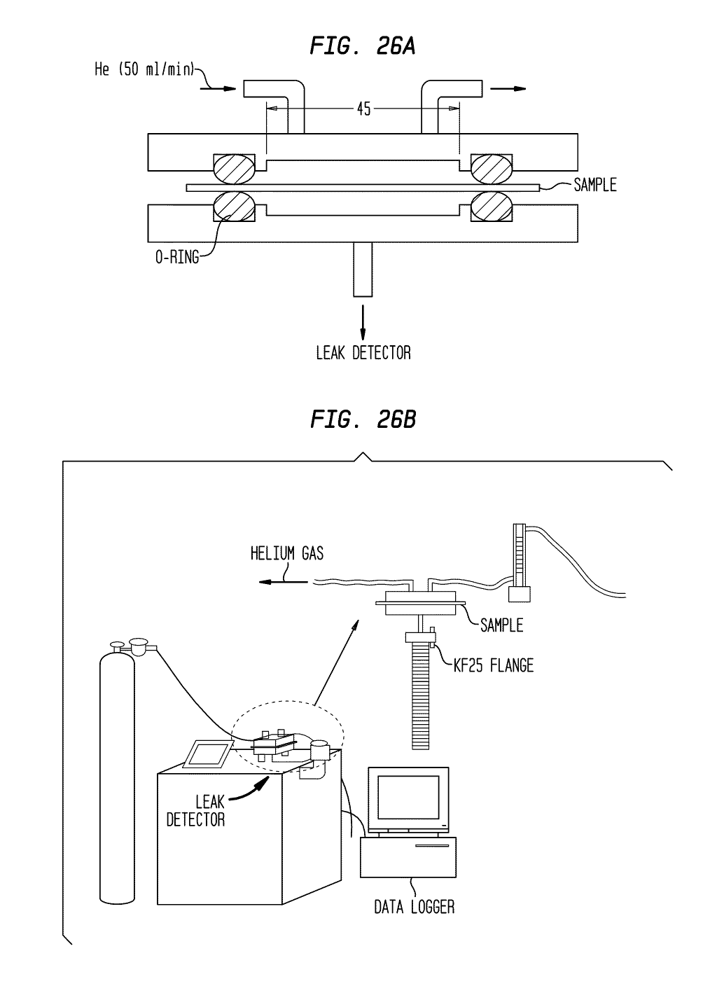

Two specific examples of plasma activation (i.e., the deposition of two initial surface coatings on PEEK) will be assessed: tetrahedral amorphous carbon (ta-C) or titanium (Ti). Amorphous and semi-crystalline PEEK films of 250 micron thickness will be coated with either ta-C or Ti, with thicknesses of 5, 10, 50 or 100 nm.
The permeation of helium gas through the coated polymer films will be measured using a custom made permeability chamber. Helium will flow through the chamber and the time taken for helium to be detected on the other side of the film will be measured. A similar test apparatus as described by Ogasawara et al. and as schematically illustrated in FIGS. 26a and 26b may be used to test the gas permeation of a polymer/clay material. Hermetic capsules in accordance with embodiments of the present invention may be validated with a test that measures water vapour ingress, for example using a MOCON Aquatran. The water vapour test is used in addition to a helium test.
The study design indicating the groups and sample numbers is listed in Table 14.

TABLE 14
Details of study design

| PEEK | | Deposition | |
|---|---|---|---|
| morphology | Coating type | thickness (nm) | Sample size |
| Amorphous | Uncoated | N/A | 2 |
| | (control) | | |
| Semi-crystalline | Uncoated | N/A | 2 |
| | (control) | | |
| Amorphous | ta-C | 5, 10, 20, 50 or | 2 of each thickness |
| | | 100 | |
| Semi-crystalline | ta-C | 5, 10, 20, 50 or | 2 of each thickness |
| | | 100 | |
| Amorphous | Ti | 5, 10, 20, 50 | 2 of each thickness |
| | | or 100 | |
| Semi-crystalline | Ti | 5, 10, 20, 50 | 2 of each thickness |
| | | or 100 | |

### Hermeticity Test of Capsules

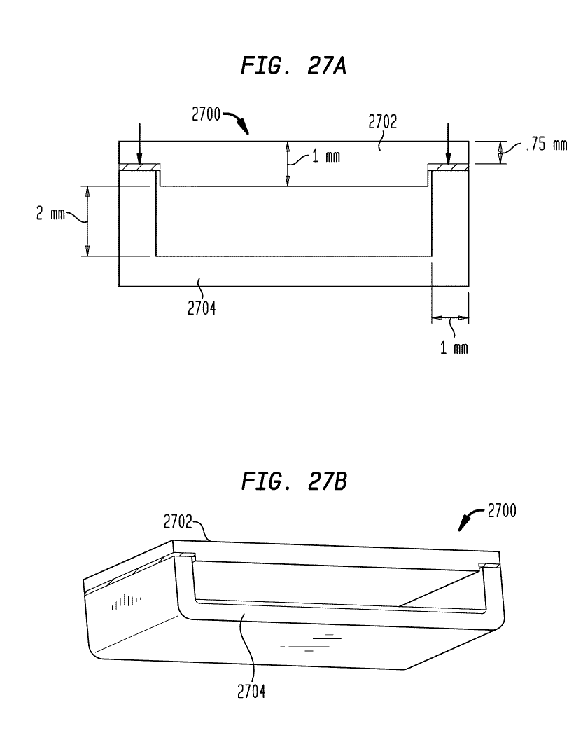

FIGS. 27a and 27b are schematic cross-sectional and perspective views, respectively, of a capsule 2700 in accordance with embodiments of the present invention. The capsule shown in FIGS. 27a and 27b will be injection molded from PEEK as two separate parts or pieces of material: lid 2702 and base 2704. The lid will be welded to the base using plasma activated direct bonding, or laser welding, or a combination of both. The hermeticity test will measure water vapor ingress into the cavity. A traditional helium “bomb test” cannot be used due to the inherent permeable nature of polymeric materials, which could result in false readings due to helium absorbed in the bulk material rather than an actual leak path through the bond.

Desiccant will be placed inside the cavity before welding. The welded capsule will then be immersed in water or saline which is maintained at a constant elevated temperature (in accordance with accelerated aqueous immersion tests). The weight of the capsule will be measured at predetermined time intervals for up to one year. This will provide information about the absorption of moisture into the polymer. At the end of each time interval, the capsule will be opened and the desiccant alone also weighed. This will provide additional information regarding how much water the polymer absorbed without entering the cavity.

### Conclusion

This protocol will provide quantitative information regarding the hermeticity of the bonding techniques (direct and laser), and an indication of the reduction of permeability using surface coating techniques.

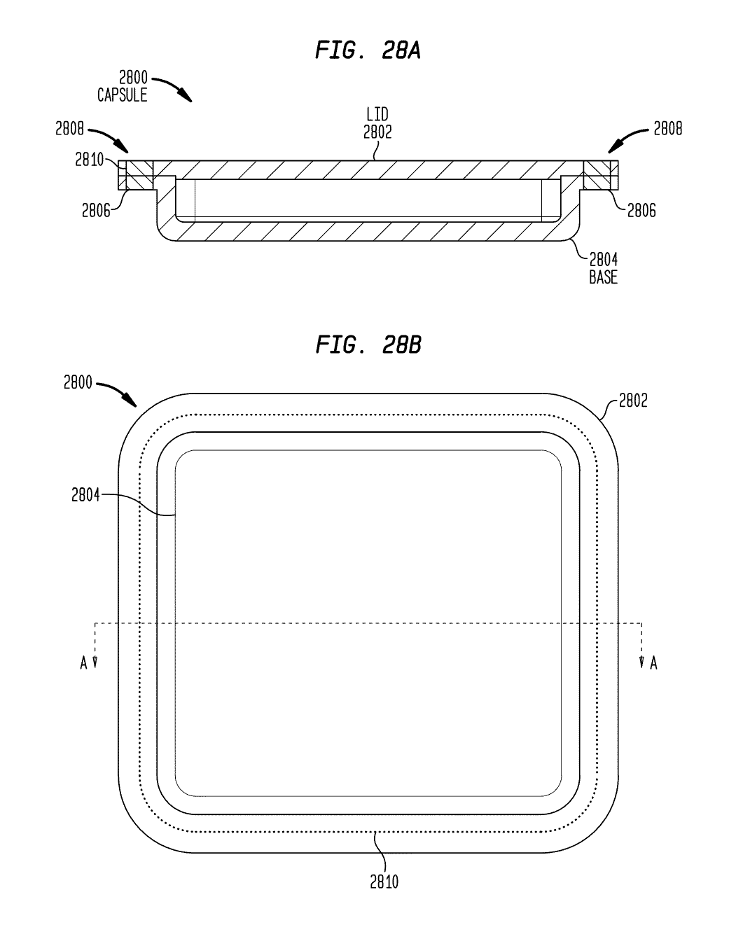

FIG. 28a is a cross-sectional view of a hermetic encapsulation capsule 2800 having an alternative joint configuration, in accordance with embodiments of the present invention. As shown, capsule 2800 comprises a polymeric lid 2802 and a polymeric base 2804. Lid 2802 and base 2804 are, in these embodiment, injection molded components formed from PEEK, and are collectively sometimes referred to herein as a PEEK-PAK.

In an exemplary method of the present invention, lid 2802 and base 2804 are each treated using radio-frequency (RF) excited argon plasma with ion implantation. In this embodiment, lid 2802 and base 2804 are placed on the sample stage and are covered by an electrically connected mesh. The system is evacuated to base pressure of 5.0×10⁻⁵ Torr and then high purity argon gas is introduced into the chamber with a flow rate of 38.6 cm³/min. The operating power of the RF power supply is approximately 125 W, with 25 W reflected power. High voltage pulses are applied to the sample holder after a stable argon RF plasma is sustained. The working pressure is approximately 4.2×10⁻³ Torr. The implantation voltage, pulse width and pulse repetition rate are approximately 10 kV, 25 μs and 2000 Hz, respectively. The surfaces of lid 2802 and base 2804 are PIII treated for approximately 2 min.

One or more components are placed in base 2804 before lid 2802 is attached thereto. In one embodiment, lid 2802 and base 2804 are positioned into holes of a hot press tool and then placed into a temperature controlled hot press, such as a Tetrahedron hot press (Tetrahedron Inc. USA). The bonding process is carried at a temperature of approximately 200° C. under a force of approximately 0.8 ton for approximately 4 hours.
In the arrangement of FIG. 28a, base 2804 has a flange 2806 extending therefrom. The bond 2810 between lid 2802 and based 2804 is formed at flange 2806, schematically illustrated by area 2808 in FIG. 28a. FIG. 28b is a schematic top view of capsule 2800 illustrating that bond 2801 extends around the circumference of the capsule to hermetically seal the volume between bases 2804 and lid 2802. In particular, this bond may be reinforced using transmission laser welding, using parameters as provided above.

Although the above described embodiments were discussed with reference to a cochlear implant, in other embodiments these methods and systems may be used with other implant systems such as, for example, in an auditory brain stimulator or other tissue-stimulating prosthesis.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All documents, patents, journal articles and other materials cited in the present application are hereby incorporated by reference.

1. An implantable medical device, comprising:
an implantable component; and
a polymeric hermetic encapsulation disposed around the component having one or more plasma activated surfaces directly bonded to one another.

2. The device of claim 1, wherein the polymeric encapsulation comprises a polyaryletherketone (PAEK) hermetic encapsulation.

3. The device of claim 1, wherein the polymeric hermetic encapsulation comprises first and second components having abutting surfaces configured to mate with one another, and wherein portions of the abutting surfaces are plasma activated and directly bonded to one another.

4. The device of claim 2, wherein the PAEK encapsulation comprises one or more PAEK polymers selected from the group comprising:
polyetherketone (PEK), polyetheretherketone (PEEK), polyetherketoneketone (PEKK), polyetheretherketoneketone (PEEKK), polyetherketoneetherketoneketone (PEKEKK) and combinations thereof.

5. The device of claim 4, wherein the PAEK material further comprises one or more polymers selected from the group comprising:
acrylonitrile butadiene styrene, celluloid, cellulose acetate, cycloolefin copolymer, ethylene chlorotrifluoroethlyene, ethylene tetrafluoroethylene, ethylene vinyl acetate, ethylene vinyl alcohol, fluorinated ethylene propylene, ionomer, Kydex, liquid crystal polymer, MP-1, perfluoroalkoxy, polyacetal, polyacrylonitrile, polyamide, polyamide-imide, polyamide-imide-phthalmide, polybutadiene, polybutylene, polybutylene terephthalate, polycaprolactone, polycarbonate, polychlorotrifluoroethylene, polycyclohexylene dimethylene terephthalate, polyester, polyetherimide, polyethersulfone, polyethylene, polyethylenechlorinate, polyethylene terephthalate, polyhydroxyalkanoate, polyimide, polyketone, polylactic acid, polymethylmethacrylate, polymethylpentene, polyphenylene, polyphenylene oxide, polyphenylene sulfide, polyphenylene sulfone, polyphthalamide, polypropylene, polystyrene, polysulfone, polytetrafluoroethylene, polytrimethylene terephthalate, polyurethane, polyvinyl acetate, polyvinyl chloride, polyvinylidene chloride, styrene-acrylonitrile and combinations thereof.

6. The device of claim 1, wherein the polymeric encapsulation comprises a fluorinated polymer.

7. The device of claim 6, wherein the fluorinated polymer is polyterafluoroethlene.

8. The device of claim 1, wherein the plasma activation of the one or more polymeric surfaces enhances one or more of interdiffusion, entanglement, bridging and radical diffusion during direct bonding.

9. The device of claim 1, wherein the plasma activation of the one or more polymeric surfaces enhances crystallization of the polymer chains during directed bonding.

10. The device of claim 1, wherein the plasma activation induces molecular reactions in the one or more polymer surfaces, including scission, oxidation, nitration, crosslinking and/or condensation of polymer chains.

11. The device of claim 1, wherein the plasma activation involves plasma immersion ion implantation (PIII).

12. The device of 1, wherein the one or more polymer surfaces are exposed to surface deposition at least one of before, during and after the plasma activation.

13. The device of claim 1, wherein the one or more polymer surfaces are exposed to laser welding at least one of before, during and after the plasma activation.

14. The device of claim 1, wherein the medical device is an active implantable device selected from the group comprising a hearing prosthesis, a heart stimulating or assistance device, a nerve stimulating or detection device or a body process stimulating or monitoring device.

15. The device of claim 1, wherein the encapsulation has a permeability to water vapor of less than approximately 0.1 mg/m²/day.

16. A method of hermetic encapsulating a component of an implantable medical device with a polymeric material, comprising:
exposing one or more surfaces of the polymeric material to plasma activation;
positioning the polymeric material around the component such that portions of the one or more activated surfaces are abutting one another; and
directly bonding the abutting portions of the one or more activated surfaces to one another to hermetically seal the component within the material.

17. The method of claim 16, wherein directly bonding the abutting portions of the one or more activated surfaces to one another comprises:
applying heat to the abutting portions of the surfaces.

18. The method of claim 17, wherein directly bonding the abutting portions of the one or more activated surfaces to one another comprises:
applying pressure to the abutting portions of the surfaces.

19. The method of claim 16, further comprising:
laser welding one or more of the polymeric surfaces at least one of before, during and after the plasma activation.

20. The method of claim 16, wherein the polymeric material comprises a first component having a volume therein to receive the component, and wherein second component comprises a element configured to mate with the first component, and wherein portions of the surfaces of the first and second components are exposed to plasma activation, the method comprises:
mating the plasma activated surfaces of the first and second components; and
directly bonding the mated surfaces to one another.

21. The method of claim 16, wherein the polymeric material comprises polyaryletherketone (PAEK).

22. The method of claim 20, wherein the PAEK material comprises one or more PAEK polymers selected from thee group comprising:
polyetherketone (PEK), polyetheretherketone (PEEK), polyetherketoneketone (PEKK), polyetheretherketoneketone (PEEKK), polyetherketoneetherketoneketone (PEKEKK) and combinations thereof.

23. The method of claim 21, wherein the PAEK material further comprises one or more polymers selected from the group comprising: acrylonitrile butadiene styrene, celluloid, cellulose acetate, cycloolefin copolymer, ethylene chlorotrifluoroethlyene, ethylene tetrafluoroethylene, ethylene vinyl acetate, ethylene vinyl alcohol, fluorinated ethylene propylene, ionomer, Kydex, liquid crystal polymer, MP-1, perfluoroalkoxy, polyacetal, polyacrylonitrile, polyamide, polyamide-imide, polyamide-imide-phthalmide, polybutadiene, polybutylene, polybutylene terephthalate, polycaprolactone, polycarbonate, polychlorotrifluoroethylene, polycyclohexylene dimethylene terephthalate, polyester, polyetherimide, polyethersulfone, polyethylene, polyethylenechlorinate, polyethylene terephthalate, polyhydroxyalkanoate, polyimide, polyketone, polylactic acid, polymethylmethacrylate, polymethylpentene, polyphenylene, polyphenylene oxide, polyphenylene sulfide, polyphenylene sulfone, polyphthalamide, polypropylene, polystyrene, polysulfone, polytetrafluoroethylene, polytrimethylene terephthalate, polyurethane, polyvinyl acetate, polyvinyl chloride, polyvinylidene chloride, styrene-acrylonitrile and combinations thereof.

24. The method of claim 17, 18 or 19, wherein exposing one or more surfaces of the polymeric material to plasma activation includes:
plasma immersion ion implantation (Pm).

25. The method of claim 17, 18 or 19, further comprising:
exposing one or more surfaces of the polymeric material to surface deposition at least one of before, during and after the plasma activation to further reduce the permeability of the material.

26. The method of claim 16, wherein the positioning the polymeric material around the component comprises:
positioning the polymeric material around a component of an active implantable device selected from the group comprising: a hearing prosthesis, a heart stimulating or assistance device, a nerve stimulating or detection device or a body process stimulating or monitoring device.

27. A hermetically sealed device formed through the method of claim 16.
